(19) United States
(12) Patent Application Publication
(10) Pub. No.: US 2019/0117590 A1
(43) Pub. Date: Apr. 25, 2019

(54) Compositions and Methods for Treating Hyperproliferative Disorders

(71) Applicant: Texas Tech University System, Lubbock, TX (US)

(72) Inventors: Barry James Maurer, Idalou, TX (US); Charles Patrick Reynolds, Lubbock, TX (US)

(21) Appl. No.: 16/227,335

(22) Filed: Dec. 20, 2018

Related U.S. Application Data
(63) Continuation of application No. 12/945,808, filed on Nov. 12, 2010, now abandoned.
(60) Provisional application No. 61/260,595, filed on Nov. 12, 2009.

(51) Int. Cl. A61K 31/133; A61K 31/5375; A61K 31/131; A61K 31/167

(52) U.S. Cl. CPC A61K 31/133; A61K 31/167; A61K 31/131; A61K 31/5375

(57) ABSTRACT

A method of treating a hyperproliferative disorder in a subject in need of such treatment, comprising administering to said subject, in combination, a treatment effective amount of: (a) a ceramide-increasing retinoid such as fenretinide or a pharmaceutically acceptable salt thereof; and (b) at least one (and in certain embodiments at least two) compounds selected from the groups consisting of (i) a non-18 carbon chain length L-threo-sphinganine(s) or pharmaceuticeutically acceptable salt thereof, (ii) glucosylceramide or glucosyl(dihydro)ceramide synthesis inhibitor(s), and (iii) sphingomyelin or dihydrosphingomyelin synthase inhibitor(s). Preferred L-threo-sphinganines are of carbon chain length 17 carbons, 19 carbons and 20 carbons. A preferred glucosylceramide or glucosyl(dihydro)ceramide synthesis inhibitor IS D-threo-1-phenyl-2-palmitoylamino-3-morpholino-1-propanol. A preferred sphingomyelin or dihydrosphingomyelin synthesis inhibitor is D-threo-1-phenyl-2-palmitoylamino-3-morpholino-1-propanol. A preferred hyperproliferative disorder is brain cancers.

### CROSS-REFERENCE TO RELATED APPLICATIONS

This Application claims the benefit under Title 35 United States Code§ 119(e) of U.S. Provisional Application No. 61/260,595, filed Nov. 12, 2009, the full disclosure of which is incorporated herein by reference.

### FIELD OF THE INVENTION

The present invention relates to the combination of novel. sphingoid bases and their use in chemotherapy regimens for the treatment of hyperproliferative disorders.

### BACKGROUND OF THE INVENTION

Without limiting the scope of the disclosed method, the background is described in connection with a novel approach in the treatment of hyperproliferative disorders.

Sphinganines constitute a group of related long-chain aliphatic 2-amino-1,3-diols of which 0-erythro-sphinganine (i.e, D-erythro-dihydrosphingosine=(2S,3R)-2-aminooctadecanel,3-diol=0-erythro-2-amino-1,3-octadecanediol=(2S,3R)-2-amino-1,3-octadecanediol), an 18 carbon length sphinganine, is the most frequent naturally-occurring sphinganine in mammals. 20-carbon chain length 0-erythro-sphinganine is also used in limited quantities in mammals and generally restricted to the central nervous system. Sphinganines are converted into dihydroceramides by acylation of their C2-amino group with fatty acids of varying chain length, generally 14 carbon to 30 carbon chain length. Dihydroceramides are further converted into ceramides by desaturation of the bond between Carbons 4 and 5 of the sphinganine backbone, i.e., the placement of a carbon-carbon double bond. Ceramides are predominantly used to make higher order sphingolipids, i.e. waxes, for the manufacture and repair of cellular membranes, and as signaling molecules. Historically, the term “ceramides” refers to both dihydroceramides and ceramides. The Carbon 4,5 double bound also distinguishes sphinganines from sphingosines. Historically, sphinganines and sphingosines are collectively referred to as “sphingoid bases”. All naturally-occurring mammalian sphinganines and sphingosines are of D-erythro stereochemistry regarding the chirality of the C2-carbon amino group and C3-carbon hydroxyl group.

Safingol is the artificial, L-threo-stereochemical (diastereomer) variant of native, 18 carbon chain length, D-erythro-sphinganine. Safingol is also variously named L-threosphinganine=L-threo sphinganine (2S, 3S)=L-threo-dihydrosphingosine=L-threo-2-amino-1,3-octadecanediol=(2S,3S)-2-amino-1,3-octadecanediol. Safingol has been reported to increase the anticancer activity of the retinoid (Vitamin A-derivative), fenretinide.

Fenretinide [HPR; all-trans-N-(4-hydroxyphenyl) retinamide; CAS Registry number 65646-68-6] is currently believed to effect cytotoxicity in cancer cells by generating reactive oxygen species and by increase of dihydroceramides. See, e.g., D. Delia et al., Carcinogenesis 18, 943-948 (1997); N. Oridate et al., J Nat/. Cancer Inst. 89, 1191-1198 (1997).

U.S. Pat. No. 4,665,098 to Gibbs describes pharmaceutical compositions of fenretinide as useful for the treatment of breast and bladder cancer.

U.S. Pat. No. 7,169,819 to Gupta et al. describes pharmaceutical compositions of fenretinide suitable for the treatment of hyperproliferative disorders, including cancers.

U.S. Pat. No. 5,821,072 to Schwartz et al. provides methods for screening protein kinase C inhibitors, including safingol, capable of potentiating apoptosis in tumor cells, along with methods for screening antitumor therapeutic agents suitable for combination therapy with a protein kinase C inhibitor capable of potentiating apoptosis in tumor cells.

U.S. Pat. No. 6,352,844 to Maurer et al. provides for a method of treating hyperproliferative disorders, including cancers, by treating a patient in need of treatment with a ceramide-generating retinoid, such as fenretinide, with safingol.

The present invention proposes a novel method for treating hyperproliferative disorders.

### SUMMARY OF THE INVENTION

The present invention, therefore, provides a method to treat hyperproliferative disorders.

The present invention relates to L-threo-sphinganine compositions of non-18 carbon chain length (i.e. exclusive of safingol) represented by the following Formula I:

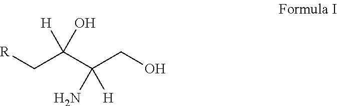

(wherein R=a linear, saturated C6-26 hydrocarbon chain, excluding a 14-carbon chain length, and wherein compositions are of L-threo stereochemistry, i.e., of (2S,3S) configuration regarding the C2-amino and C3-hydroxyl groups).

Examples of such L-threo-sphinganine compositions include, but are not limited to, Lthreo-C20-sphinganine, also called, L-threo-icosasphinganine =L-threo-eicosasphinganine=Lthreo-2-amino-1,3-icosanediol=(2S,3S)-2-amino: 1,3-icosanediol=L-threo-2-amino-1,3-eicosediol (2S,3 S)-2-amino-1,3-eicosediol (2S,3S)-2-amino-1,3-dihydroxy-eicosane (2S,3S)-2-amino-1,3-dihydroxyeicosane;

L-threo-C 19-sphinganine, also called, L-threo-2-amino-1,3-nonadecanediol=(2S,3S)-2-amino-I ,3-nonadecanediol; and L-threo-C 17-sphinganine, also called, L-threo-2-amino-1,3-heptadecanediol=(2S,3S)-2-amino-1,3-heptadecanediol.

The present invention further relates to the unexpected discovery that such L-threosphinganines increase the anticancer properties of fenretinide in human cancer cell lines. Thus, the activity against hyperproliferative disorders, such as cancers, of fenretinide and other such retinoic acid derivatives that increase ceramides (i.e. dihydroceramides or ceramides) can be enhanced by administering in proximity such L-threo-sphinganines. Such administration can be sequentially, with the L-threo-sphinganine(s) administered prior to the ceramides-increasing retinoid or fenretinide, or concurrently, with the L-threo-sphinganine administered during part or all of the ceramides-increasing retinoid or fenretinide administration period, or with the L threosphinganine administered after the ceramides-increasin¥ retinoid or fenretinide, so long as the beneficial effect is realized. The present invention also relates to the treatment of hyperproliferative disorders wherein such L-threo-sphinganine(s) are administered with a ceramides-increasing retinoid or fenretinide as described, together with an additional agent that manipulates cellular metabolism and cellular control of ceramide-generated cytotoxicity (e.g., a ceramide degradation inhibitor). Such agents include glucosylceramide and glucosyl(dihydro)ceramide synthase inhibitors and sphingomyelin and (dihydro)sphingomyelin synthase inhibitors, and which may be administered alone or in combination with one another. Specific examples are given below. Preferably, the retinoic acid derivative or fenretinide is given in an amount effective to produce necrosis, apoptosis, autophagy, or other death-inducing process in the tumor cell, and the L-threo-sphinganine, with or without the ceramides degradation inhibitor, is given in an amount effective to increase the necrosis, apoptosis, authophagy or other death-inducing process in the tumor cell over that which would be produced by the retinoic acid derivative or fenretinide alone, or that expected to be produced by the sum of the retinoic acid derivative or fenretinide and the L-threo-sphinganine with or without the ceramide degradation inhibitor when given separately.

The present invention also concerns a method of treating a hyperproliferative disorder in a subject in need of such treatment comprises administering to the subject, in combination, a treatment effective amount of: (a) a retirioic acid derivative that increases dihydroceramides or ceramides, such as fenretinide or a pharmaceutically acceptable salt thereof; and (b) a non-18 carbon chain length L-threo-sphinganine(s) or pharmaceutically acceptable salt thereof and, optionally, (c) a glucosylceramide or glucosyl(dihydro)ceramide synthesis inhibitor (including the pharmaceutically acceptable salts thereof), such as D-threo-1-phenyl-2-palmitoylamino-3- morpholino-1-propanol or a pharmaceutically acceptable salt thereof, and optionally, (d) a sphingomyelin or (dihydro)sphingomyelin synthase inhibitor. The synthesis inhibitor(s) is administered in an amount effective to enhance the activity of the retinoic acid derivative and the

L-threo-sphinganine, such that compounds together have an efficacious activity. Preferably, the retinoic acid derivative and L-threo-sphinganine are given in an amount effective to produce necrosis, apoptosis, or autophagy other cell death process in the tumor cell, and the synthesis inhibitor is given in an amount effective to increase the necrosis, apoptosis, or autophagy or other cell death process produced in the tumor cell over that which would be expected by the retinoic acid derivative and L-threo-sphinganine combined, or that expected to be produced by the sum of the retinoic acid derivative and L-threo-sphinganine combination and the synthesis inhibitor when given separately. Other compounds including the compounds described herein may also be administered.

A theory of action is that the beneficial effect of L-threo-sphinganines on ceramide increasing retinoids in human and canine hyperproliferative disorders is that such retinoids increase cellular D-erythro-dihydroceramides in susceptible hyperproliferative disorders, such as cancers, while L-threo-sphinganines are metabolically converted into L-threo-dihydroceramides to effect their beneficial actions. It is not concluded that the beneficial effect will be observed in all mammals, such as rodents, in which L-threo-sphinganines are metabolically converted into Lthreo- ceramides. It is not excluded that L-threo-sphinganines performs a function(s) contributory to the function of the present invention that is distinct from its conversion into L threodihydroceramides. Therefore, non-18 carbon chain length L-threo-sphinganines, and other compounds which perform this function(s), are active in the present invention and included therein, without binding applicants to a particular underlying theory of the invention. Virtually all mammalian sphingolipids are made using C-18 and C-20 carbon length sphingoid backbones, and therefore it would not be expected that non-C 18 and C20 sphinganines, of any stereochemistry, would function at all. Further, C-20 backbone sphingolipids are not found outside the CNS in any appreciable quantity, and therefore it would not be expected that C-20-Lthreo- sphinganines would function as is in fact observed as demonstrated herein.

Formulations comprising portions of the aforesaid combinations of compounds in a single pharmaceutical carrier or vehicle, for carrying out the foregoing treatments, are also an aspect of the instant invention. The use of the foregoing compounds for the preparation of a medicament for carrying out the aforesaid treatments are also an aspect of the instant invention.

The foregoing is illustrative of the present invention, and is not to be construed as limiting thereof. The invention is defined by the following claims, with equivalents of the claims to be included therein. In summary, the present invention discloses an improved method for treating a hyperprolifertive disorder.

### BRIEF DESCRIPTION OF THE DRAWINGS

For a more complete understanding of the present invention, reference is now made to the detailed description of the invention along with the accompanying figures in which:

FIG. 1 is the dose-response of C17-L-threo-sphinganine in combination with fenretinide showing cytotoxicity at one or more doses in the drug-resistant CHLA-90 neuroblastoma cancer cell line.

FIG. 2 is the dose-response of C17-L-threo-sphinganine in combination with fenretinide showing cytotoxicity at one or more doses in the CHLA-266 brain cancer (PNET) cell line in 2% oxygen.

FIG. 3 is the dose-response of C17-L-threo-sphinganine in combination with fenretinide showing cytotoxicity at one or more doses in the GBM2 glioblastoma brain cancer cell line.

FIG. 4 is the dose-response of C17-L-threo-sphinganine in combination with fenretinide showing cytotoxicity at one or more doses in the HT-29 colon cancer cell line in 2% oxygen.

FIG. 5 is the dose-response of C17-L-threo-sphinganine in combination with fenretinide showing cytotoxicity at one or more doses in the COG-LL-317 Acute Lymphoblastic Leukemia (ALL) cancer cell line in 5% oxygen.

FIG. 6 is the dose-response of C 17-L-threo-sphinganine in combination with fenretinide showing cytotoxicity at one or more doses in the MOLT-4 ALL leukemia cell line.

FIG. 7 is the dose-response of C 19-L-threo-sphinganine in combination with fenretinide showing cytotoxicity at one or more doses in drug resistant the CHLA-90 neuroblastoma cancer cell line.

FIG. 8 is the dose-response of C 19-L-threo-sphinganine in combination with fenretinide showing cytotoxicity at one or more doses in the CHLA-266 brain cancer (PNET) cell line in 2% oxygen.

FIG. 9 is the dose-response of C 19-L-threo-sphinganine in combination with fenretinide showing cytotoxicity at one or more doses in the GBM2 glioblastoma brain cancer cell line.

FIG.10 is the dose-response of C19-L-threo-sphinganine in combination with fenretinide showing cytotoxicity at one or more doses in the HT-29 colon cancer cell line.

FIG. 11 is the dose-response of C19-L-threo-sphinganine in combination with fenretinide showing cytotoxicity at one or more doses in the COG-LL-317 ALL leukemia cell line in 5% oxygen.

FIG. 12 is the dose-response of C20-L-threo-sphinganine in combination with fenretinide showing cytotoxicity at one or more doses in the drug resistant CHLA-90 neuroblastoma cell line.

FIG. 13 is the dose-response of C20-L-threo-sphinganine in combination with fenretinide showing cytotoxicity at one or more doses in the CHLA-266 brain cancer (PNET) cell line in 2% oxygen.

FIG. 14 is the dose-response of C20-L-threo-sphinganine in combination with fenretinide showing cytotoxicity at one or more doses in the GBM2 glioblastoma brain cancer cell line.

FIG. 15 is the dose-response of C20-L-threo-sphinganine in combination with fenretinide showing cytotoxicity at one or more doses in the HT-29 colon cancer cell line.

FIG. 16 is the dose-response of C20-L-threo-sphinganine in combination with fenretinide showing cytotoxicity at one or more doses in the MCF-7/ADR (OVCAR-8/ADR) ovarian cancer cell line in 2% oxygen.

FIG. 17 is the dose-response of C20-L-threo-sphinganine in combination with fenretinide showing cytotoxicity at one or more doses in the COG-LL-317 ALL cell line in 5% oxygen.

FIG. 18 is the dose-response of C17-L-threo-sphinganines in combination with fenretinide and D-threo-PPMP showing synergistically increased cytotoxicity at most doses in the GBM2 brain cancer cell line in 2% oxygen.

FIG. 19 is the dose-response of C19-L-threo-sphinganines in combination with fenretinide and D-threo-PPMP showing synergistically increased cytotoxicity at most doses in the GBM2 brain cancer cell line in 2% oxygen.

FIG. 20 is the dose-response of C20-L-threo-sphinganines in combination with fenretinide and D-threo-PPMP showing synergistically increased cytotoxicity at most doses in the GBM2 brain cancer cell line in 2% oxygen.

FIG. 21 is the dose-response of C17-L-threo-sphinganines in combination with fenretinide and D-threo-PPMP showing synergistically increased Cytotoxicity at most doses in the HT-29 colon cancer cell line in 20% oxygen.

FIG. 22 is the dose-response of C 19-L-threo-sphinganines in combination with fenretinide and D-threo-PPMP showing synergistically increased cytotoxicity at most doses in the HT-29 colon cancer cell line in 20% oxygen.

FIG. 23 is the dose-response of C20-L-threo-sphinganines in combination with fenretinide and D-threo-PPMP showing synergistically increased cytotoxicity at most doses in the HT-29 colon cancer cell line in 2% oxygen.

FIG. 24 is the dose-response of C 17-L-threo-sphinganines in combination with fenretinide and D-threo-PPMP showing synergistically increased cytotoxicity at most doses in the MOLT-4 ALL leukemia cell line in 2% oxygen.

FIG. 25 is the dose-response of C19-L-threo-sphinganines in combination with fenretinide and D-threo-PPMP showing synergistically increased cytotoxicity at most doses in the MOLT-4 ALL leukemia cell line in 2% oxygen.

FIG. 26 is the dose-response of C20-L-threo-sphinganines in combination with fenretinide and D-threo-PPMP showing synergistically increased cytotoxicity at most doses in the MOLT-4 ALL leukemia cell line in 2% oxygen.

FIGS. 27A-27D show the results of Fenretinide and L-threo-sphinganines tested in normal human fibroblast (normal skin cell) cell lines, CRL-2091 and CRL-2076.

FIGS. 28A-28D show the results of Fenretinide and L-threo-sphinganines tested in human Multiple Myeloma (a cancer of the blood and bone marrow) cell line, RPMI-8226.

FIGS. 29A-29D show the results of Fenretinide and L-threo-sphinganines tested in human Multiple Myeloma (a cancer of the blood and bone marrow) cell line, U-266.

FIGS. 30A-30D show the results of Fenretinide and L-threo-sphinganines tested in human Glioblastoma multiforme (brain cancer) cell line, A-172.

FIGS. 31A-31D show the results of Fenretinide and L-threo-sphinganines tested in human Glioblastoma (brain cancer) cell line, U-118.

FIGS. 32A-32D show the results of Fenretinide and L-threo-sphinganines tested in human Glioblastoma multiforme (brain cancer) cell line, T98G.

FIGS. 33A-33D show the results of Fenretinide and L-threo-sphinganines tested in human Glioblastoma multiforme (brain cancer) cell line, SJ-GBM2.

FIGS. 34A-34D show the results of Fenretinide and L-threo-sphinganines tested in human Glioblastoma (brain cancer) cell line, SJ-G2.

FIGS. 35A-35B show the results of Fenretinide and L-threo-sphinganines tested in human primitive neuroectodermal tumor (PNET) (brain cancer) cell line, CHLA-266.

FIGS. 36A-36D show the results of Fenretinide and L-threo-sphinganines tested in human colorectal adenocarcinoma (colon cancer) cell line, HT-29.

FIGS. 37A-37D show the results of Fenretinide and L-threo-sphinganines tested in human melanoma (skin cancer) cell line, A-2058.

FIGS. 38A-38B show the results of Fenretinide and L-threo-sphinganines tested in human lung adenocarcinoma (lung cancer) cell line, NCI-H-1792.

FIGS. 39A-39D show the results of Fenretinide and L-threo-sphinganines tested in human lung adenocarcinoma (lung cancer) cell line, A-549.

FIGS. 40A-40D show the results of Fenretinide and L-threo-sphinganines tested in human breast adenocarcinoma (breast cancer) cell lines, MCF-7 and MDA-MB-231.

FIGS. 41A-41D show the results of Fenretinide and L-threo-sphinganines tested in human ovarian adenocarcinoma (ovarian cancer) cell line, OVCAR-8.

FIGS. 42A-42B show the results of Fenretinide and L-threo-sphinganines tested in human prostate adenocarcinoma (prostate cancer) cell line, LNCaP.

FIGS. 43A-43D show the results of Fenretinide and L-threo-sphinganines tested in human prostate adenocarcinoma (prostate cancer) cell line, PC-3.

FIGS. 44A-44D show the results of Fenretinide and L-threo-sphinganines tested in human pancreatic adenocarcinoma (pancreas cancer) cell line, PANC-1.

FIGS. 45A-45D show the results of Fenretinide and L-threo-sphinganines tested in human esophageal adenocarcinoma (esophagus cancer) cell lines, OE-19 and OE-33.

FIGS. 46A-46D show the results of Fenretinide and L-threo-sphinganines tested in human acute lymphoblastic leukemia (pediatric ALL, a blood cancer) cell lines, COG-LL-317 and MOLT-4.

FIGS. 47A-47B show the results of Fenretinide and L-threo-sphinganines tested in human pediatric neuroblastoma (a nerve-related, solid tumor cancer) cell line, CHLA-90.

### DETAILED DESCRIPTION OF THE INVENTION

Disclosed herein are novel sphingoid bases and their use in combination chemotherapy regimens for the treatment of hyperproliferative disorders. The numerous innovative teachings of the present invention will be described with particular reference to several embodiments (by way of example, and not of limitation).

The disclosed combination of novel sphingoid bases and their use in chemotherapy regimens for the treatment of hyperproliferative disorders is generally described, with examples incorporated as particular embodiments of the invention and to demonstrate the practice and advantages thereof. It is understood that the examples are given by way of illustration and are not intended to limit the specification or the claims in any manner.

The methods of the present invention utilize the combined effects of retinoic acid derivatives, such as fenretinide, coupled with the novel sphingoid bases described herein as potentiating agents, to manipulate cellular metabolism and cellular control of ceramide-generated toxicity, in order to inhibit or prevent the growth of tumors, cancers, neoplastic tissue and other premalignant and noneoplastic hyperproliferative disorders, all of which are together referred to as hyperproliferative or hyperplastic disorders herein. The treatments employed herein may be used to inhibit growth and/or to induce cytotoxicity (by necrotic or apoptotic mechanisms, or both) in the target cells, which are generally hyperproliferative cells (including tumors, cancers, and neoplastic tissue, along with pre-malignant and non-neoplastic or non-malignant hyperproliferative disorders).

Examples of tumors, cancers, and neoplastic tissue that can be treated by the present invention include but are not limited to malignant disorders such as breast cancers; osteosarcomas; angiosarcomas; fibrosarcomas and other sarcomas; leukemias; lymphomas;

sinus tumors; ovarian, cervical, ureteral, bladder, prostate and other genitourinary cancers; colon, esophageal and stomach cancers and other gastrointestinal cancers; lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas.

Examples of premalignant and non-neoplastic or non-malignant hyperproliferative disorders include but are not limited to myelodysplastic disorders; cervical carcinoma-in-situ; familial intestinal polyposes such as Gardner syndrome; oralleukoplakias; histiocytoses; keloids; hemangiomas; hyperproliferative arterial stenosis, inflammatory arthritis; hyperkeratoses and papulosquamous eruptions including arthritis. Also included are viral induced hyperproliferative diseases such as warts and EBV-induced disease (i.e., infectious mononucleosis), scar formation, and the like. The methods of treatment disclosed herein may be employed with any subject known or suspected of carrying or at risk of developing a hyperproliferative disorder as defined herein.

As used herein, “treatment” of a hyperproliferative disorder refers to methods of killing, inhibiting or slowing the growth or increase in size of a body or population of hyperproliferative cells or tumor or cancerous growth, reducing hyperproliferative cell numbers, or preventing spread to other anatomic sites, as well as reducing the size of a hyperproliferative growth or numbers of hyperpproliferative cells. As used herein, “treatment” is not necessarily meant to imply cure or complete abolition of hyperproliferative growths. As used herein, a treatment effective amount is an amount effective to result in the killing, the slowing of the rate of growth of hyperproliferative cells, the decrease in size of a body of hyperproliferative cells, and/or the reduction in number of hyperproliferative cells. The potentiating agent (or agents) is included in an amount sufficient to enhance the activity of the first compound, such that the two (or more) compounds together have greater therapeutic efficacy than the individual compounds given alone (e.g., due to synergistic interaction; reduced combined toxicity, etc.).

As used herein, the administration of two or more compounds “in combination” means that the two compounds are administered closely enough in time that the presence of one alters the biological effects of the other. The two compounds may be administered simultaneously (concurrently) or sequentially. Simultaneous administration may be carried out by mixing the compounds prior to administration, or by administering the compounds at the same point in time but at different anatomic sites or using different routes of administration.

The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

Subjects to be treated by the methods of the present invention include both human subjects and animal subjects for veterinary purposes. Animal subjects are preferably mammalian subjects including horses, cows, dogs, cats, rabbits, sheep, and the like.

A variety of intracellular molecules are known to trigger or inhibit cell death (S. Rowan and D. Fisher, Leukemia 11, 457 (1997); K. Saini and N. Walker, Mol. Cell Biochem. 178, 9 (1998)). Most current work focuses on elucidating pathways for programmed cell death (apoptosis), in which triggers of apoptosis (such as DNA damage) can activate various pathways
(e.g. p53, Fas, and others), which can be modulated by yet other molecules (such as the Bcl-2 family of pro-and anti-apoptotic proteins), with caspase activation being a late step in the final events leading to apoptotic cell death. However, not all cell death occurs via apoptosis, and cell death induced by 4-HPR involves both apoptosis and necrosis (J. Clifford et al., Cancer Res. 59,
14 (1999)) or autophagy (Zheng, W., et al., Biochem Biophys Acta. 1758:1864-84 (2006), Tiwari, M., et al., Carcinogenesis 29:600-9, (2008), Fazi, B., et al., Autophagy.4:435-41, (2008). The intracellular lipid ceramide is known to mediate apoptosis (L. Obeid et al., Science 259, 1769 (1993)(FIG. 1) and necrosis (Guo et al., Am. J. Physiol. 276, F390 (1999); Condorelli et al., Br. J. Pharmacol. 137, 75 (1999)). It has been shown to cause the apoptosis-inducing permeability transition of mitochondrial membranes (S. Susin et al., J. Exp. Med. 186, 25 (1997)), cause apoptosis-inducing ROS generation by mitochondrial complex III inhibition (A.
Quillet-Mary et al., J. Biol. Chem. 272, 21388 (1997) and activate the pro-death JNK/SAPK pathway (S. Basu et al., Oncogene 17, 3277 (1998); T. Okazaki et al., Cell. Signal. 10, 685 (1998); W. Jarvis, Curr. Opin. Oncol. 10, 552 (1998)). Ceramide also activates a protein kinase (CAPK) (S. Mathias et al., Biochem. J. 335(Pt 3), 465 (1998) and a phosphorylase (PP2A) (L.
Leoni et al., Biochem. Pharmacol. 55, 1105 (1998)) and can lead to the activation of the nuclear transcription factor, NF-kappaB (L. Johns et al., J. Immunol. 152, 5877 (1998); C. Garnard et al., J. Biol. Chem. 272, 1682 (1997)). Mechanisms by which cancer cells avoid the cytotoxic effects of ceramide can include metabolism to other forms, including nontoxic glucosylceramide (Y. La vie et al., J. Biol. Chem. 272, 1682 (1997); Y. La vie et al., J. Biol. Chem. 271, 19530 (1996); L. Yong-Yu et al., J. Biol. Chem. 274, 1140 (1999)) and sphingosine- 1-phosphate. Sphingosine-1-phosphate opposes ceramide-induced cell death by activating the pro-life ERK 1/2 pathway (O. Cuvillier et al., Nature 381, 800 (1996); O. Cuvillier et al., J. Biol. Chem. 273, 2910 (1998)). Thus, modulation of ceramide metabolism offers a means for enhancing the cytotoxic efficacy of 4-HPR (fenretinide) and other ceramide-generating retinoids.

Ceramide is generated intracellularly via activation of ceramide synthase, the de novo synthetic pathway or by activation of the neutral- or acidic- sphingomyelinases, leading to breakdown of sphingomyelin. Cerarnide is metabolized to non-cytotoxic glucosylceramide by glucosylceramide synthase; and converted into cytotoxic sphingosine by alkaline- or acidicceramidases. Sphingosine is further converted to the anti-apoptotic sphingosine-1-phosphate by sphingosine kinase. We show below that modulation of these pathways can enhance, even synergistically enhance, the cytotoxicity of ceramide-generating retinoids such as 4-HPR (fenretinide).

Compounds that may be used to carry out the present invention, and formulations thereof and the manner of administering the same, are described in detail below.

1. Ceramide-Generating Retinoids.

Ceramide-generating retinoids or retinoic acid derivatives that can be used to carry out the present invention are those generating ceramide in a host cell to which they are administered and include those described in U.S. Pat. No. 4,190,594 to Gander (the disclosures of all patent references cited herein are incorporated herein by reference). Ceramide-generating retinoids include all trans-retinoic acid (A TRA) and retinoic acid derivatives, including but not limited to:

(A) esters of all-trans-retinoic acid having the following Formula II:

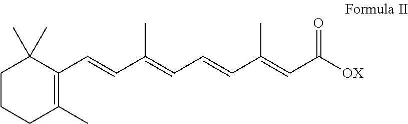

wherein X is a member selected from the group consisting of (Formulas III &

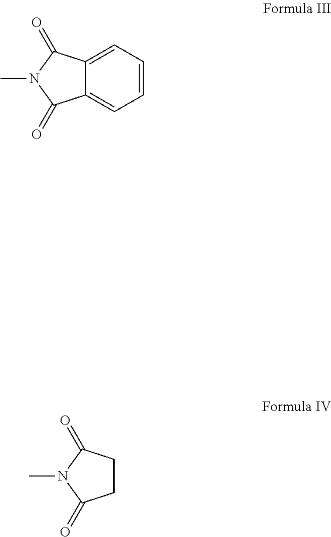

2-cyclohexylethyl; 1 0-carbomethoxydecyl; 4-hydroxybutyl; cholesteryl; mixed m- and p-vinylbenzyl; and 4-bromobenzyl;

(B) esters ofall-trans-retinoic acid having the following Formula V:

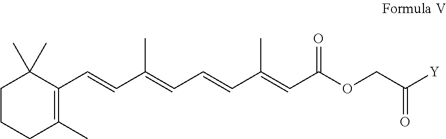

wherein Y is a member selected from the group consisting of: cholesteryloxy; phenyl; 4 bromophenyl; 4-methoxyphenyl;, 4-nitrophenyl; 4-hydroxyphenyl; 4-methylphenyl; 4-cyanophenyl; 4-ethoxyphenyl; 4-acetoxyphenyl; 2-naphthyl; 4-biphenyl; 2,5-dimethoxyphenyl; 2,4-dichlorophenyl; 2,4-dimethylphenyl; 3,4-diacetoxyphenyl; 3,4,5-trimethoxyphenyl; and 2,4,6-trimethylphenyl; and

(C) am ides of all-trans-retinoic acid having the following Formula VI:

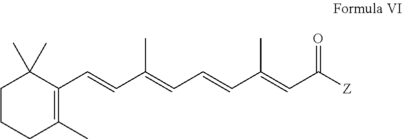

wherein Z is a member selected from the group consisting of: n-propylamino; tertbutylamino; 1,1 ,3,3-tetramethylbutylamino; 1-morpholino; 4-hydroxypheny˜amino; 4-carbomethoxy-2-hydroxyphenyl amino; beta-(3,4-dimethoxyphenyl)-ethylamino; 2-benzothiazolylamino; 1-imidazolyl; 1-(2-nicotinoylhydrazolyl); 1-benzotriazolyl; 1-(1 ,2,4-triazolyl) (Formulas VII, VIII & IX); and

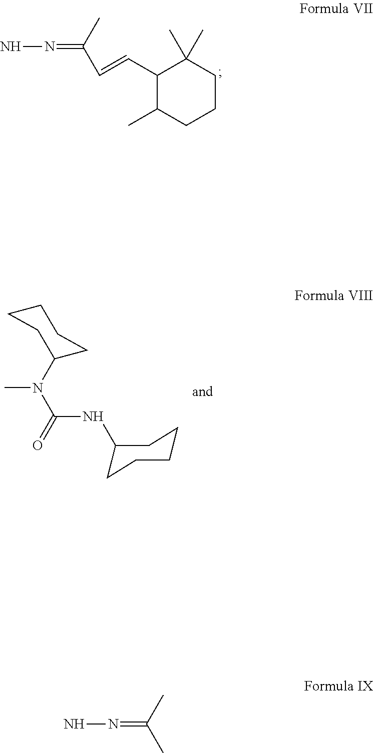

Particularly preferred is all-trans-N-(4-hydroxyphenyl)retinamide, also called fenretinide, which has CAS registry number 65646-68-6, and has the structure (Formula X):

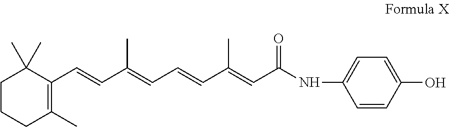 [SMILES: CC1=C(/C=C/C(C)=C/C=C/C(C)=C/C(=O)Nc2ccc(O)cc2)C(C)(C)CCC1]

The foregoing compounds can be prepared in accordance with known techniques. See, e.g., U.S. Pat. No.4,190,594 to Gander et al.; U.S. Pat. No. 4,665,098 to Gibbs.

Additional retinoic acid derivatives that can be used to carry out the present invention include C-Glycoside analogs of N-(4-hydroxyphenyl)retinamide-0-glucuronide. Such compounds and their preparation are known and described in U.S. Pat. Nos. 5,663,377 and 5,599,953, both to Curley et al., the disclosures of which are incorporated by reference herein in their entirety. Such compounds may have the general formula (Formula XI):

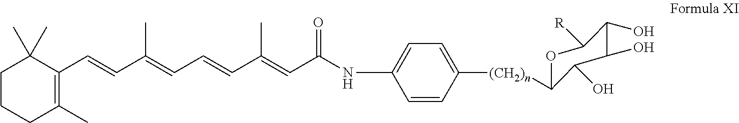

where R is COOH, CH.sub.2 OH, or H, and n is 0 or 1.

Specific examples of such compounds include: 4-(retinamido)phenyl-C-glucuronide; 4- (retinamido)phenyl-C-glucoside; 4-(retinamido)phenyi-C-xyloside; 4-(retinamido)benzyi-C-glucuronide; 4-(retinamido)benzyl-C-glucoside; 4-(retinamido)benzyi-C-xyloside; 1-(.beta.-D-glucopyranosyl) retinamide; and 1-(D-glucopyranosyluronosyl) retinamide.

2. Glucosylceramide Synthesis Inhibitors.

Any compound that inhibits glycosylceramide or glycosyl(dihydro)ceramide synthesis can be used, particularly glucosylceramide synthase inhibitors. Examples of such compounds include, but are not limited to, compounds having the formula (Formula XII):

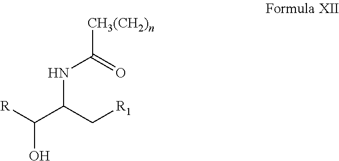

where R is an aromatic ring such as phenyl, a cyclohexyl group, or an alpiphatic group having 10 to 15 carbon atoms, R.sub.l is an amine group such as a morpholino group; and n is an integer of from 4 to 18 (including functional homologues, isomers and pharmaceutically acceptable salts thereof. Preferably, n is 4, 6, 8, I 0, 12 or 14, and the D enantiomer of such compounds are preferred. Such compounds are known and are disclosed, for example, in U.S. Pat. No. 5,302,609 to Shayman and Radin; U.S. Pat. No. 5,041,441 to Radin et al.; and U.S. Pat. No. 5,707,649 to Inokuchi et al. Specific examples of glucosylceramide synthase inhibitors include: 1-phenyl-2-acylamino-3-morpholino-1-propanol in which n is 6 to 12; 1-phenyl-2- decanoylamino-3-morpholino-1-propanol (POMP); and 1-phenyl-2-palmitoylamino-3- morpholino-1-propanol (PPMP);

3. Additional Active Compounds and Screening.

Additional active compounds can be generated by known techniques, including rational drug design techniques and/or random drug design techniques (or combinatorial chemistry techniques).

In active compounds that interact with a receptor, the interaction takes place at the surface-accessible sites in a stable three-dimensional molecule. By arranging the critical binding site residues in an appropriate conformation, compounds which mimic the essential surface features of the active compound binding region may be designed and synthesized in accordance with known techniques. A molecule which has a surface region with essentially the same molecular topology to the binding surface of the active compound will be able to mimic the interaction of the active compound with its corresponding receptor. Methods for determining the three-dimensional structure of active compounds and producing active analogs thereof are known, and are referred to as rational drug design techniques. See, e.g., U.S. Pat. No. 5,593,853 to Chen; U.S. Pat. Nos. 5,612,895 and 5,331,573 to Balaji et al.; U.S. Pat. No. 4,833,092 to Geysen; U.S. Pat. No. 4,859,765 to Nestor; U.S. Pat. No. 4,853,871 to Pantoliano; and U.S. Pat.

No. 4,863,857 to Blalock (the disclosures of all U.S. Patent references cited herein are to be incorporated herein by reference).

In combinatorial chemistry (or random drug design) techniques, large combinatorial libraries of candidate compounds are screened for active compounds therein. Libraries used to carry out the present invention may be produced by any of a variety of split synthesis methods. Split synthesis methods in which a releasable tag is attached to the particle along with the organic compounds of interest are also known as cosynthesis methods. A variety of such methods are known. See, e.g., A. Furka et al., J. Pept. Protein Res. 37,487 (1991); K. Lam et al., Nature 354, 82 (1991); R. Zuckermann et al., Int. J. pept. Protein Res. 40,498 (1992); F. Sebestyen et al., Bioorg. Med. Chem. Lett. 3, 413 (1993); K. Lam et al., Bioorg. Med. Chem. Lett. 3, 419 (1993). For example, the library may be a library of organometallic compounds wherein the compound is a metal-ligand complex. The metal in the complex may be an early or late transition metal in high, low or zero oxidation states. The metal may also be any of the main group metals, alkali metals, alkaline earths, lanthanides or actinides. The ligand in the metal-ligand complex may be composed of, or derived from, chiral or achiral forms of cyclopentadienes, amino esters, oxazolidoinones, hydroxy acids, hydroxy esters, hydroxy amides, pyridines, fused pyridines, nitrogen heterocycles, oxazoles, imidazoles, pyrroles, crown ethers, cryptands, carcerands, phosphines, diphosphines, polyphosphines, quinuclidines, quinines, alkaloids, dextrins, cyclodextrins, salens, porpyrins, biaryls, sulfonamides, Schiff bases, metallocenes, monools, diols, polyols, amines, diamines, polyamines, ammonium salts, peptides, proteins, nucleic acids, etc.

As a second example, the library may be a library of non-metal compounds including, but not limited to, chiral or achiral forms of cyclopentadienes, amino esters, oxazolidinones, hydroxy acids, hydroxy esters, hydroxy amides, pyridines, fused pyridines, nitrogen heterocycles, oxazoles, imidazoles, pyrroles, crown ethers, cryptands, carcerands, phosphines, diphosphines, polyphosphines, quinuclidines, quinines, alkaloids, dextrins, cyclodextrins, salens, porphyrins, biaryls, sulfonamides, Schiff bases, metallocenes, monools, diols, polyols, amines, diamines, polyamines, ammonium salts, peptides, proteins, nucleic acids, etc.

The solid supports may be separate from one another, or may be discreet regions on a surface portion of a unitary substrate, which surface portion may be positioned at the interface so that a plurality of the discreet regions are positioned at the interface. Such “chiptype” or “pin-type” solid supports are known. See, e.g., U.S. Pat. No. 5,288,514 to Ellman (pinbased support); U.S. Pat. No. 5,510,270 to Fodor et al. (chip-based support). Separate discreet supports (e.g., particles or beads) are currently preferred. Synthesis of the catalyst library and linking thereof to the discreet solid support may be carried out in accordance with known techniques, such as described in U.S. Pat. No. 5,565,324 (the disclosure of which is incorporated by reference herein in its entirety), or variations thereof that will be apparent to those skilled in the art.

Compounds selected by any means, including but not limited to those described above, may be screened for activity in increasing, including additively and synergistically increasing but preferably synergistically increasing, the cytostatic or cytotoxic activity of a ceramide-generating retinoid in tumor cells (or other hyperproliferative cells), by a method comprising: (a) contacting first control tumor cells with an amount of ceramide-generating retinoid (e.g., an amount that may or may not itself be effective to inhibit growth of said tumor cells); (b) contacting second control tumor cells with an amount of a test compound (eg., an amount that may or may not itself be effective to inhibit growth of said tumor cells); and (c) contacting experimental tumor cells with both said amount of ceramide generating retinoid in step (a) above and said amount of a test compound in step (b) above; and (d) determining the growth inhibition of said tumor cells of steps (a), (b) and (c) above; and then (e) comparing the growth inhibition or cytotoxic activity in the experimental tumor cells of step (c) with the growth inhibition of the control tumor cells of steps (a) and (b), a greater degree of growth inhibition determined in the experimental tumor cells of step (c) than the combined growth inhibition of the control tumor cells of steps (b) and (c) indicating that the test compound enhances the activity of the ceramide-generating retinoid.

The comparing step may be carried out by any suitable means, such as by calculating a Combination Index, where a value less than 1 (e.g., less than 0.9) indicates the compounds are synergistic. Any tumor cells can be used, including but not limited to neuroblastoma, lung, melanoma, prostate, leukemia, colon, breast, and pancreas tumor cells. Any ceramide-generating retinoid such as fenretinide can be used. Other hyperproliferative cells including pre-malignant and non-malignant cells can be used instead of tumor cells, as noted with respect to conditions for treatment above. In preferred embodiments, the test compound is a ceramide-degradation inhibitor, or other agent that manipulates cellular metabolism or cellular control of ceramide-generated cytotoxicity. The determining step may be carried out by looking for growth inhibition or cytotoxicity in general, or by particularly determining necrosis, apoptosis, or both. The method may be used to identify active compounds that are ceramide degradation inhibitors, other compounds that manipulate cellular metabolism or cellular control of ceramide-generated cytotoxicity, or compounds that operate by still other mechanisms in addition to those described herein.

Compounds (including the pharmaceutically acceptable salts thereof) that have not previously been known as useful in a method of treating hyperproliferative diseases in combination with a ceramide-generating retinoid, can be prepared, formulated and used in the methods described herein in addition to, or in alternative to, the ceramide-degradation inhibitors described herein. Depending upon the compounds selected for screening, such compounds may be novel compounds, may be known compounds but not previously known for a medicinal or pharmaceutical use, may be compounds previously known for a medicinal or pharmaceutical use but not previously known for use in combination with a ceramide-generating retinoid as described herein.

4. Formulations and Administration.

The active compounds described above may be formulated for administration in a single pharmaceutical carrier or in separate pharmaceutical carriers for the treatment of a variety of conditions. In the manufacture of a pharmaceutical formulation according to the invention, the active compounds including the physiologically acceptable salts thereof, or the acid derivatives of either thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.5% to 95% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will dep˜nd on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or nonaqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral or vaginal administration conveniently comprise sterile aqueous preparations of the active compound, which preparations are preferably isotonic with the blood of the intended recipient. These preparations may be administered by means of subcutaneous, intravenous, intramuscular, or intradermal injection. Such preparations may conveniently be prepared by admixing the compound with water or a glycine buffer and rendering the resulting solution sterile and isotonic with the blood.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include vaseline, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered·aqueous solution of the active compound. Suitable formulations comprise citrate or bistris buffer (pH 6) or ethanol/water and contain from 0.1 to 0.2M active ingredient.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any one active agent, the use of which is in the scope of present invention, will vary somewhat from compound to compound, patient to patient, and will depend upon factors such as the condition of the patient and the route of delivery. Such dosages can be determined in accordance with routine pharmacological procedures known to those skilled in the art, particularly in light of the disclosure provided herein.

For fenretinide, for systemic treatment, a dose to achieve a plasma level of about 1, 2, or 3 .mu.M to 10 or 20 .mu.M will be employed; typically (for oral dosing) 50 or 100 to 500 or 1000, 2000 or 3000 mg/m.sup.2 body surface area per day.

The present invention is explained in greater detail in the following non-limiting general examples, followed by more specific examples.

### EXAMPLES A1- A26

Cytotoxicity Assay: Cytotoxicity in cell lines was determined using a fluorescence-based assay employing digital imaging microscopy (DIMSCAN)(after Fragala, et al, Mol Cancer Ther, 6:886-897, 2007). DIMSCAN quantitates viable cells which selectively accumulate fluorescein diacetate and is capable of measuring cytotoxicity over a 4-5 log dynamic range by quantifying total fluorescence per well (which is proportional to viable, clonogenic cells) after eliminating background fluorescence using digital thresholding and eosin Y quenching. Briefly, cell lines were seeded into 96-well plates in 100 L of complete culture medium (10-20% serum) per well. Cells were incubated overnight in 2%, 5% or 20% oxygen (room air) prior to addition of drugs in 50 L volumes of complete medium to various final drug concentrations in replicates of 12 wells per concentration. In assays employing D-threo-PPMP, PPMP was employed at a fixed, minimally-toxic, final concentration of 10 μM in each well. Control wells received ethanol (final concentration=0.12% 0.20%) in complete medium equivalent to the maximum final ethanol concentration of drug-treated wells. Plates were incubated at various oxygen concentrations to simulate physiological hypoxia (i.e., 2% oxygen=typical solid tumor oxygen levels; 5% bone marrow oxygen levels for leukemias; 20% room air supraphysiological oxygen for comparison with typical laboratory culture conditions). Plates were assayed at 3-4 days after initiating drug exposure depending on the growth properties of each cell line, to allow for maximum cell death and outgrowth of surviving cells. To measure cytotoxicity, FDA (stock solution of 1 mg/ml in DMSO) was added, in 50 L of complete medium per well, to a final concentration of I 0 g/ml. The plates were incubated for an additional 15-30 minutes at 37 C and then 30 L of eosin Y (0.5% in normal saline) was added per well. Total fluorescence of each well was then measured using digital imaging microscopy.

FIGS. 1 to 26 illustrate the effect (Examples A 1 through A26) of the claimed invention and were carried out using the procedures disclosed herein.

FIG. 1 is the dose-response of C17-L-threo-sphinganine in combination with fenretinide. Assayed by DimScan methodology at +96 hrs. Synergy assessed by the Combination Index Method of Chou, et al. C17-L-threo-sphinganine synergized (C. I.<I) fenretinide cytotoxicity at one or more doses in the drug-resistant CHLA-90 neuroblastoma cancer cell line;

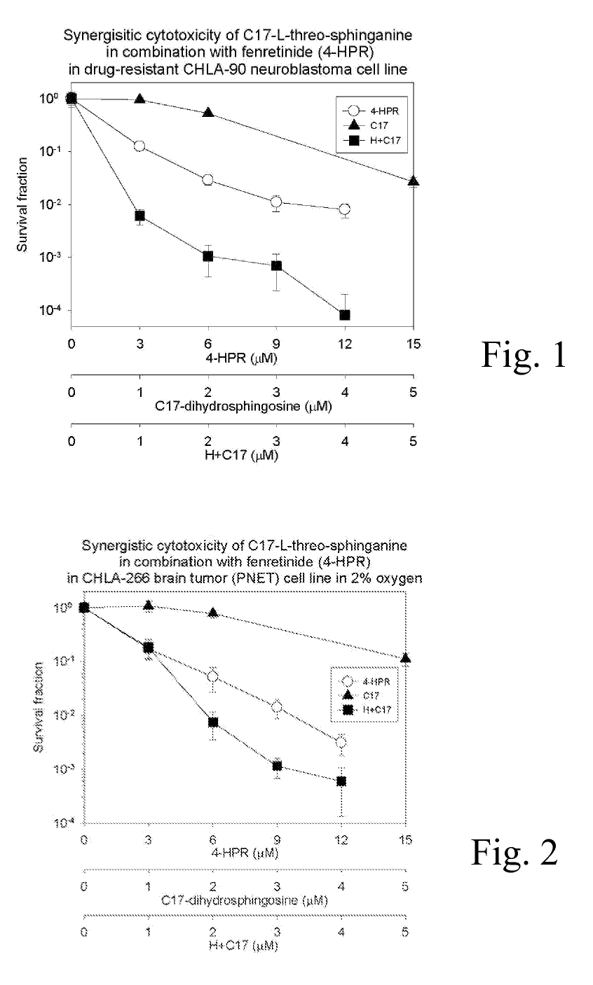

FIG. 2 is the dose-response of C17-L-threo-sphinganine in combination with fenretinide. Assayed by DimScan methodology at +96 hrs. Synergy assessed by the Combination Index Method of Chou, et al. C17-L-threo-sphinganine synergized (C.I.<1) fenretinide cytotoxicity at one or more doses in the CHLA-266 brain cancer (PNET) cell line in 2% oxygen;

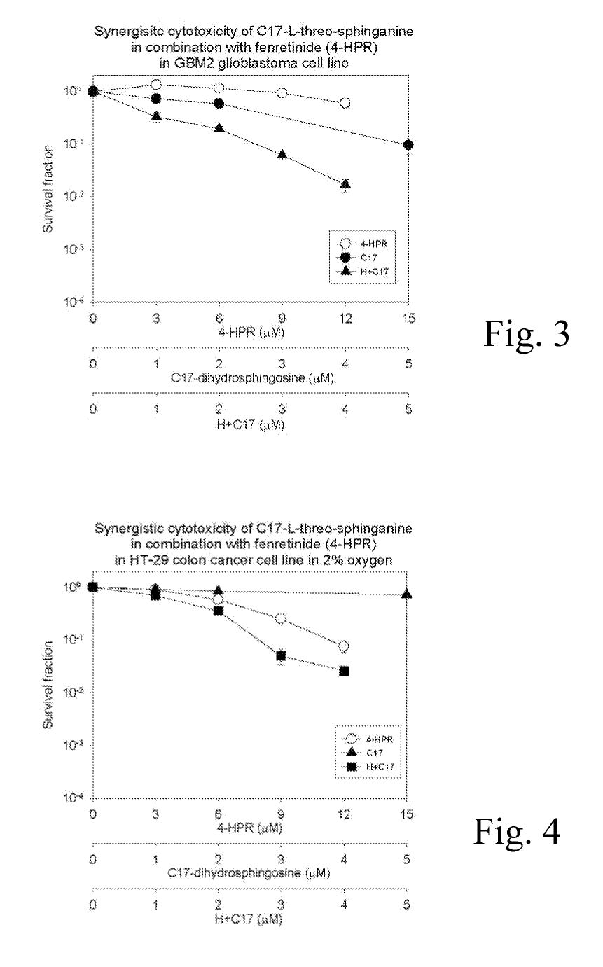

FIG. 3 is the dose-response of C17-L-threo-sphinganine in combination with fenretinide. Assayed by DimScan methodology at +96 hrs. Synergy assessed by the Combination Index Method of Chou, et al. CI7-L-threo-sphinganine synergized (C.I.<1) fenretinide cytotoxicity at one or more doses in the GBM2 glioblastoma brain cancer cell line;

FIG. 4 is the dose-response of C 17-L-threo-sphinganine in combination with fenretinide. Assayed by DimScan methodology at +96 hrs. Synergy assessed by the Combination Index Method of Chou, et al. C17-L-threo-sphinganine synergized (C.I.<1) fenretinide cytotoxicity at one or more doses in the HT-29 colon cancer cell line in 2% oxygen;

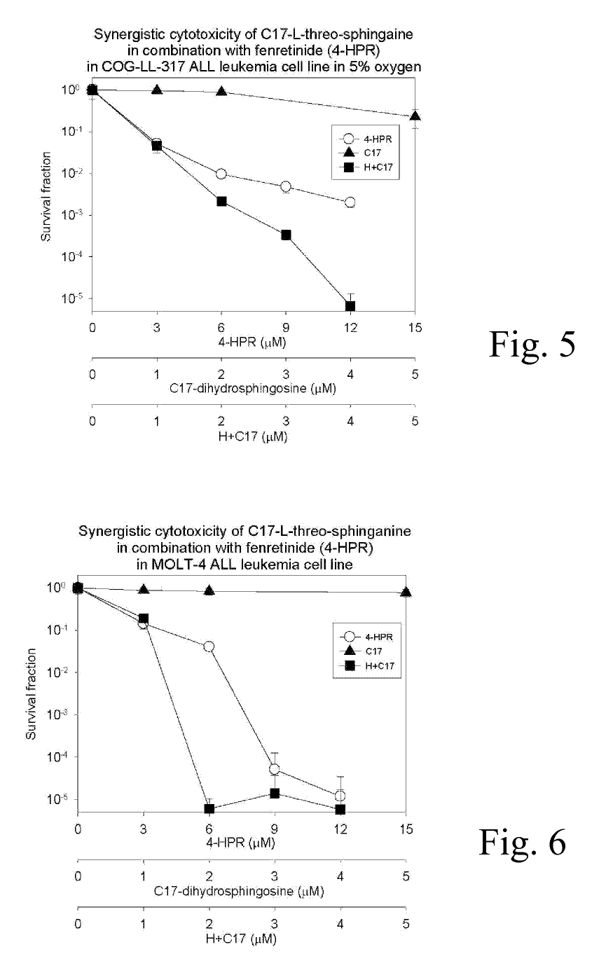

FIG. 5 is the dose-response of C17-L-threo-sphinganine in combination with fenretinide. Assayed by DimScan methodology at +96 hrs. Synergy assessed by the Combination Index Method of Chou, et al. C17-L-threo-sphinganine synergized (C.I.<1) fenretinide cytotoxicity at one or more doses in the COG-LL-317 Acute Lymphoblastic Leukemia (ALL) cancer cell line in 5% oxygen;

FIG. 6 is the dose-response of C 17-L-threo-sphinganine in combination with fenretinide. Assayed by DimScan methodology at +96 hrs. Synergy assessed by the Combination

Index Method of Chou, et al. C17-L-threo-sphinganine synergized (C.I.<1) fenretinide cytotoxicity at one or more doses in the MOLT-4 ALL leukemia cell line;

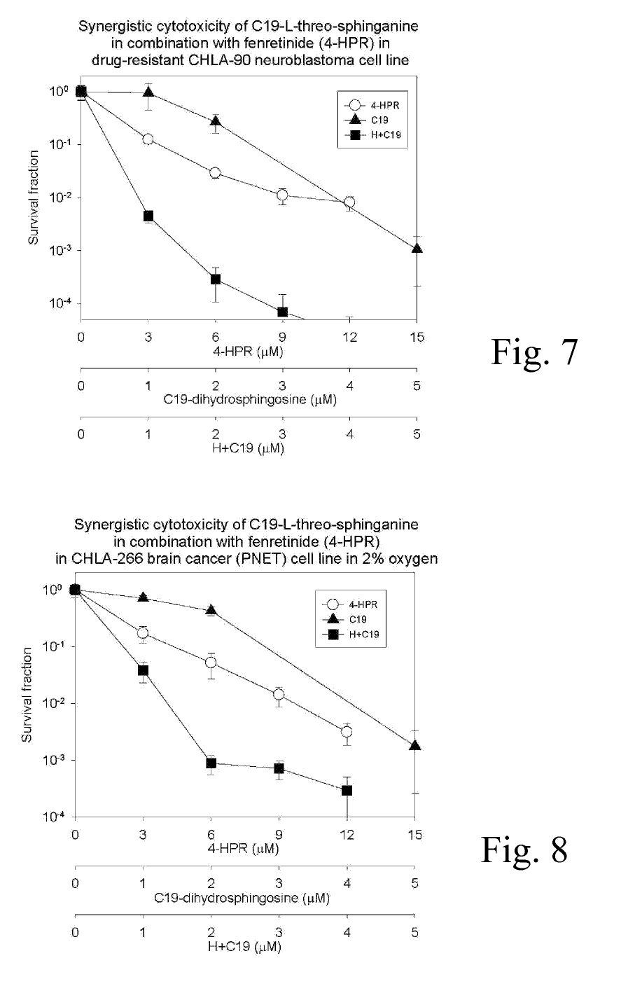

FIG. 7 is the dose-response of C 19-L-threo-sphinganine in combination with fenretinide. Assayed by DimScan methodology at +96 hrs. Synergy assessed by the Combination Index Method of Chou, et al. C19-L-threo-sphinganine synergized (C. I.<1) fenretinide cytotoxicity at one or more doses in drug resistant the CHLA-90 neuroblastoma cancer cell line;

FIG. 8 is the dose-response of C 19-L-threo-sphinganine in combination with fenretinide. Assayed by DimScan methodology at +96 hrs. Synergy assessed by the Combination Index Method of Chou, et al. C19-L-threo-sphinganine synergized (C.I.<1) fenretinide cytotoxicity at one or more doses in the CHLA-266 brain cancer (PNET) cell line in 2% oxygen;

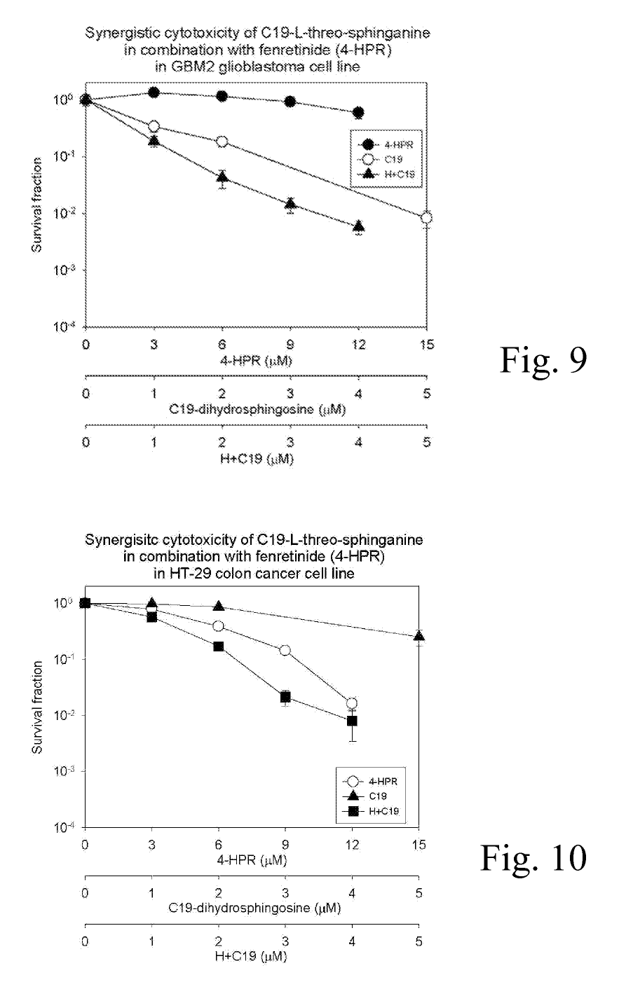

FIG. 9 is the dose-response of C19-L-threo-sphinganine in combination with fenretinide. Assayed by DimScan methodology at +96 hrs. Synergy assessed by the Combination Index Method of Chou, et al. C 19-L-threo-sphinganine synergized (C. I.<I) fenretinide cytotoxicity at one or more doses in the GBM2 glioblastoma brain cancer cell line;

FIG. 10 is the dose-response of CI9-L-threo-sphinganine in combination with fenretinide. Assayed by DimScan methodology at +96 hrs. Synergy assessed by the Combination Index Method of Chou, et al. C19-L-threo-sphinganine synergized (C.I.<1) fenretinide cytotoxicity at one or more doses in the HT-29 colon cancer cell line;

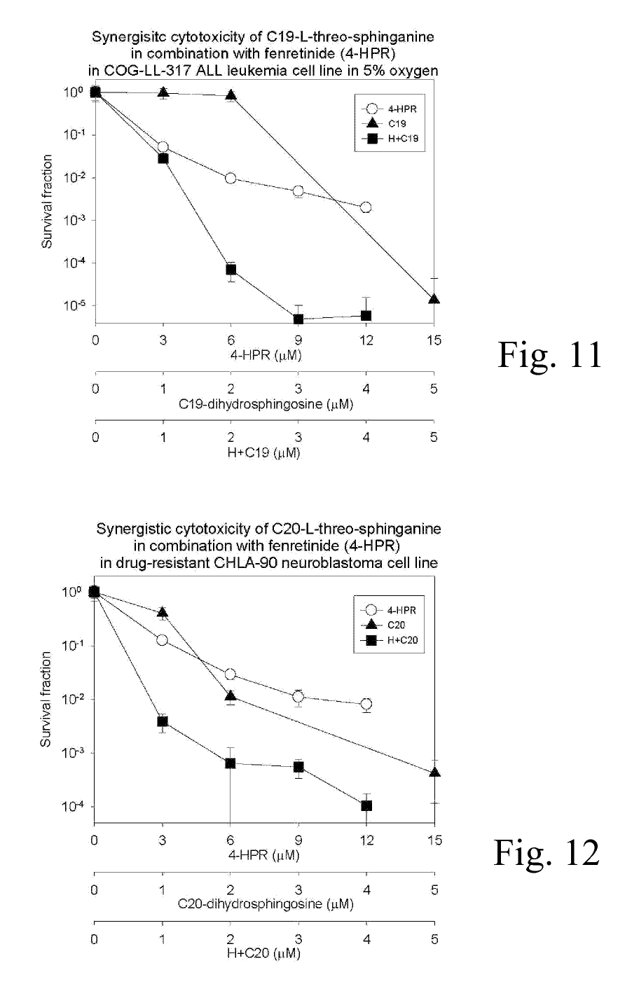

FIG. 11 is the dose-response of C19-L-threo-sphinganine in combination with fenretinide. Assayed by DimScan methodology at +96 hrs. Synergy assessed by the Combination Index Method of Chou, et al. C19-L-threo-sphinganine synergized (C.I.<I) fenretinide cytotoxicity at one or more doses in the COG-LL-3I7 ALL leukemia cell line in 5% oxygen;

FIG. 12 is the dose-response of C20-L-threo-sphinganine in combination with fenretinide. Assayed by DimScan methodology at +96 hrs. Synergy assessed by the Combination Index Method of Chou, et al. C20-L-threo-sphinganine synergized (C.I.<I) fenretinide cytotoxicity at one or more doses in the drug resistant CHLA-90 neuroblastoma cell line;

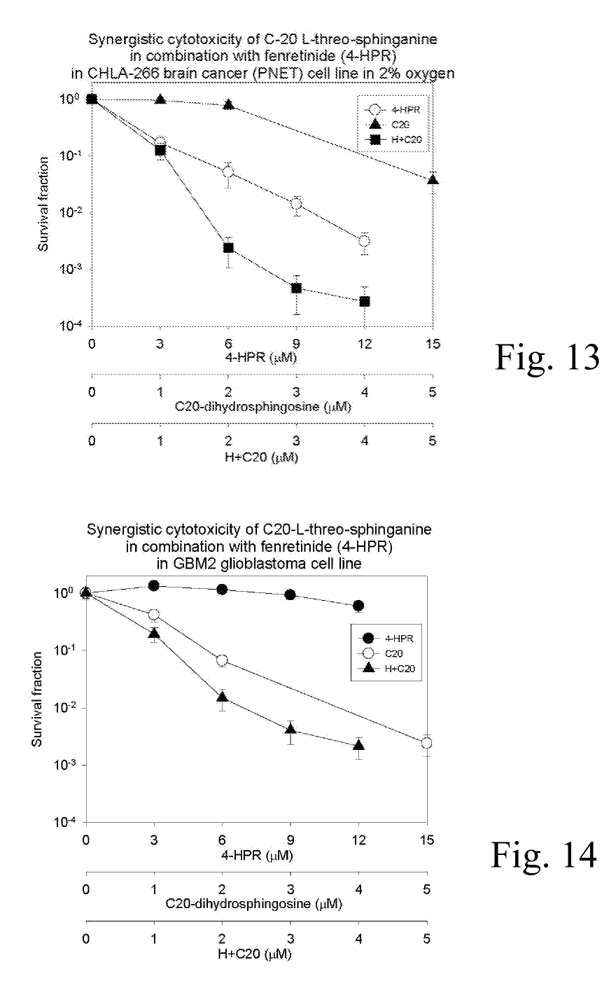

FIG. 13 is the dose-response of C20-L-threo-sphinganine in combination with fenretinide. Assayed by DimScan methodology at +96 hrs. Synergy assessed by the Combination Index Method of Chou, et al. C20-L-threo-sphinganine synergized (C.I.<1) fenretinide cytotoxicity at one or more doses in the CHLA-266 brain cancer (PNET) cell line in 2% oxygen;

FIG. 14 is the dose-response of C20-L-threo-sphinganine in combination with fenretinide. Assayed by DimScan methodology at +96 hrs. Synergy assessed by the Combination Index Method of Chou, et al. C20-L-threo-sphinganine synergized (C.I.<I) fenretinide cytotoxicity at one or more doses in the GBM2 glioblastoma brain cancer cell line;

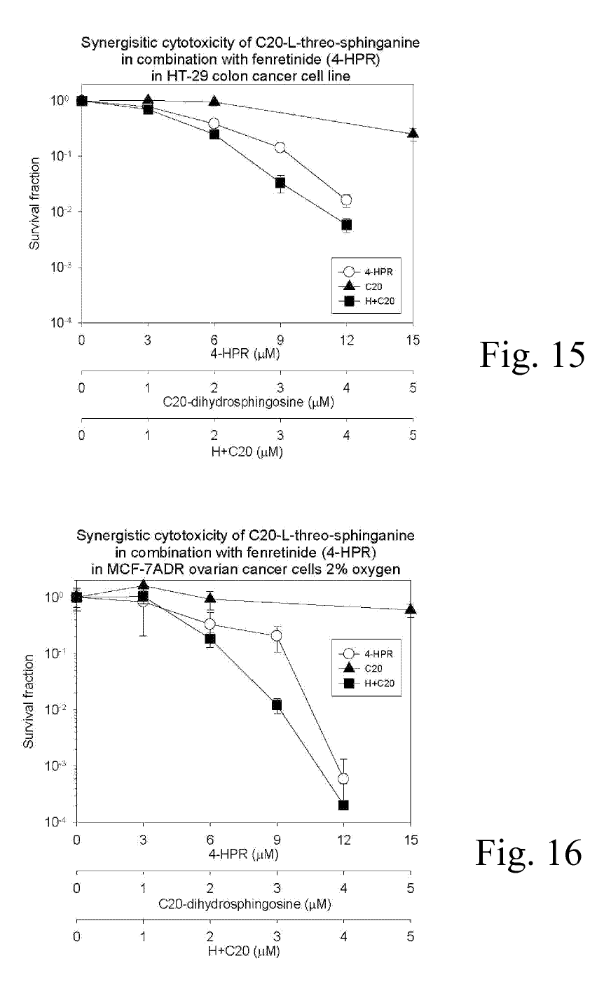

FIG. 15 is the dose-response of C20-L-threo-sphinganine in combination with fenretinide. Assayed by DimScan methodology at +96 hrs. Synergy assessed by the Combination Index Method of Chou, et al. C20-L-threo-sphinganine synergized (C.I.<I) fenretinide cytotoxicity at one or more doses in the HT -29 colon cancer cell line;

FIG. 16 is the dose-response of C20-L-threo-sphinganine in combination with fenretinide. Assayed by DimScan methodology at +96 hrs. Synergy assessed by the Combination Index Method of Chou, et al. C20-L-threo-sphinganine synergized (C.I.<I) fenretinide cytotoxicity at one or more doses in the MCF-7/ADR (OVCAR-8/ADR) ovarian cancer cell line in 2% oxygen;

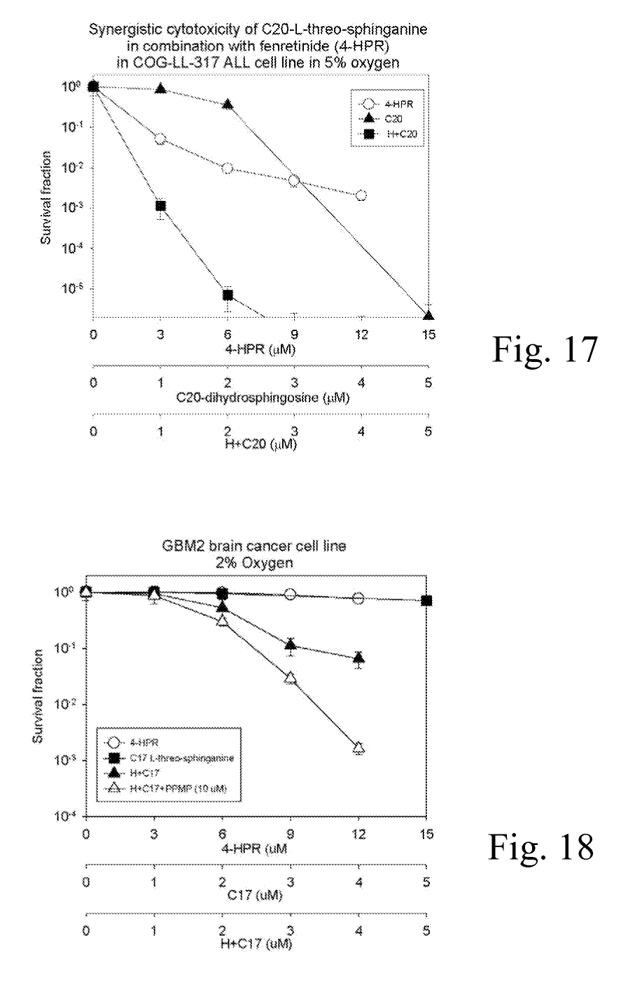

FIG. 17 is the dose-response of C20-L-threo-sphinganine in combination with fenretinide. Assayed by DimScan methodology at +96 hrs. Synergy assessed by the Combination

Index Method of Chou, et al. C20-L-threo-sphinganine synergized (C.I.<1) fenretinide cytotoxicity at one or more doses in the COG-LL-317 ALL cell line in 5% oxygen;

FIG. 18 is the dose-response of C 17-L-threo-sphinganines in combination with fenretinide and D-threo-PPMP. Assayed by DimScan methodology at +96 hrs. Three drug combination used a fixed, minimally-toxic, concentration of D-threo-PPMP (10 μM), an inhibitor of glucosylceramide synthase and sphingomyelin synthase. PPMP synergistically increased cytotoxicity (C.I.<1) of fenretinide +L-threo-sphinganine at most doses in the GBM2 brain cancer cell line in 2% oxygen by Combination Index Analysis Method of Chou, et al.;

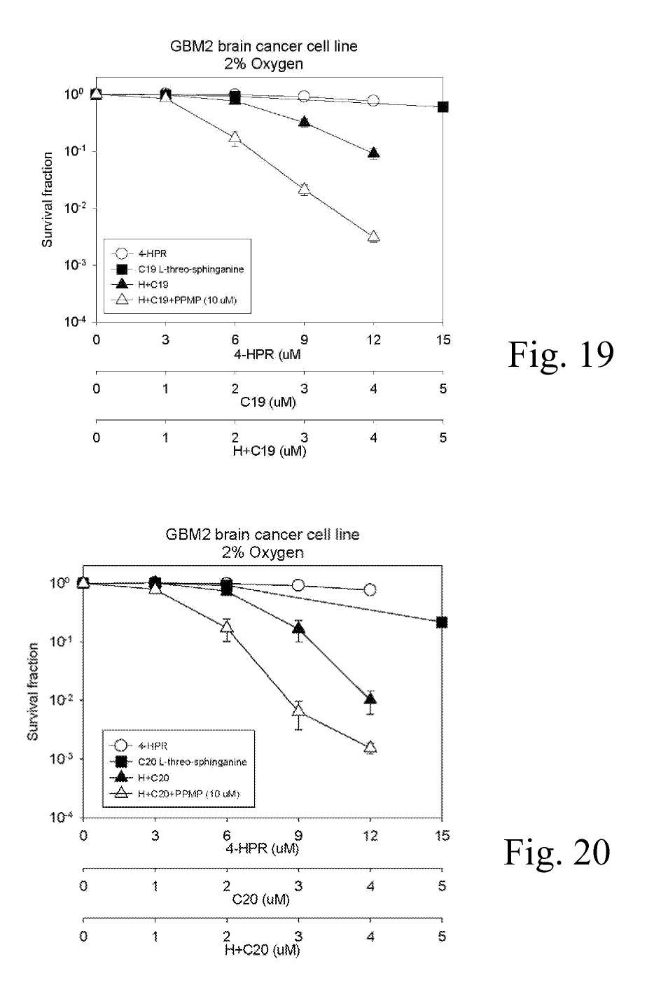

FIG. 19 is the dose-response of C19-L-threo-sphinganines in combination with fenretinide and D-threo-PPMP. Assayed by DimScan methodology at +96 hrs. Three drug combination used a fixed, minimally-toxic, concentration of D-threo-PPMP (I 0 uM), an inhibitor of glucosylceramide synthase and sphingomyelin synthase. PPMP synergistically increased cytotoxicity (C.I.<1) of fenretinide +L-threo-sphinganine at most doses in the GBM2 brain cancer cell line in 2% oxygen by Combination Index Analysis Method of Chou, et al.;

FIG. 20 is the dose-response of C20-L-threo-sphinganines in combination with fenretinide and D-threo-PPMP. Assayed by DimScan methodology at +96 hrs. Three drug combination used a fixed, minimally-toxic, concentration of D-threo-PPMP (10 μM), an inhibitor of glucosylceramide synthase and sphingomyelin synthase. PPMP synergistically increased cytotoxicity (C.I.<1) of fenretinide +L-threo-sphinganine at most doses in the GBM2 brain cancer cell line in 2% oxygen by Combination Index Analysis Method of Chou, et al.;

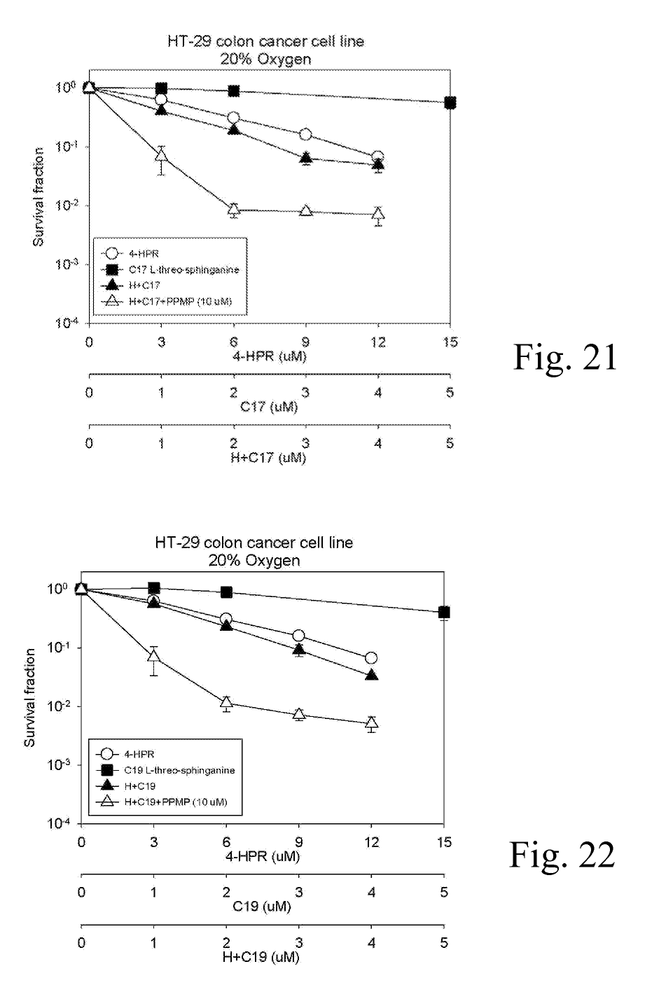

FIG. 21 is the dose-response of C17-L-threo-sphinganines in combination with fenretinide and D-threo-PPMP. Assayed by DimScan methodology at +96 hrs. Three drug combination used a fixed, minimally-toxic, concentration of D-threo-PPMP (10 uM), an inhibitor of glucosylceramide synthase and sphingomyelin synthase. PPMP synergistically increased cytotoxicity (C.I.<1) of fenretinide +L-threo-sphinganine at most doses in the HT-29 colon cancer cell line in 20% oxygen by Combination Index Analysis Method of Chou, et al.;

FIG. 22 is the dose-response of C19-L-threo-sphinganines in combination with fenretinide and D-threo-PPMP. Assayed by DimScan methodology at +96 hrs. Three drug combination used a fixed, minimally-toxic, concentration of D-threo-PPMP (10 uM), an inhibitor of glucosylceramide synthase and sphingomyelin synthase. PPMP synergistically increased cytotoxicity (C.I.<1) of fenretinide +L-threo-sphinganine at most doses in the HT-29 colon cancer cell line in 20% oxygen by Combination Index Analysis Method of Chou, et al.;

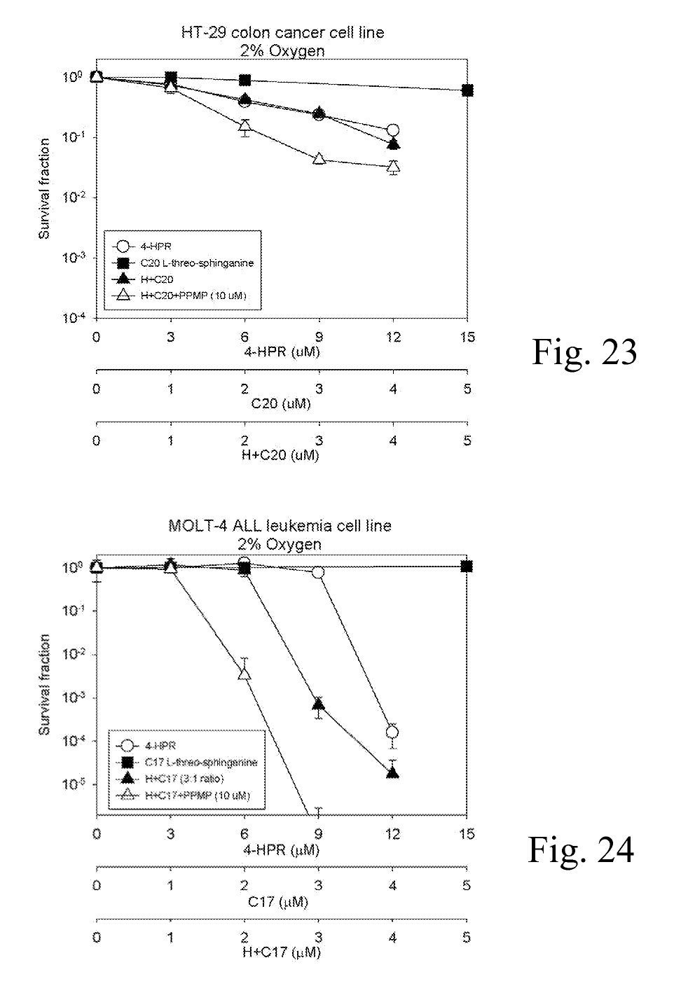

FIG. 23 is the dose-response of C20-L-threo-sphinganines in combination with fenretinide and D-threo-PPMP. Assayed by DimScan methodology at +96 hrs. Three drug combination used a fixed, minimally-toxic, concentration of D-threo-PPMP (10 uM), an inhibitor of glucosylceramide synthase and sphingomyelin synthase. PPMP synergistically increased cytotoxicity (C.I.<1) of fenretinide +L-threo-sphinganine at most doses in the HT-29 colon cancer cell line in 2% oxygen by Combination Index Analysis Method of Chou, et at.;

FIG. 24 is the dose-response of C17-L-threo-sphinganines in combination with fenretinide and D-threo-PPMP. Assayed by DimScan methodology at +96 hrs. Three drug combination used a fixed, minimally-toxic, concentration of D-threo-PPMP (10 uM), an inhibitor of glucosylceramide synthase and sphingomyelin synthase. PPMP synergistically increased cytotoxicity (C.I.<1) of fenretinide +L-threo-sphinganine at most doses in the MOLT -4 ALL leukemia cell line in 2% oxygen by Combination Index Analysis Method of Chou, et at.;

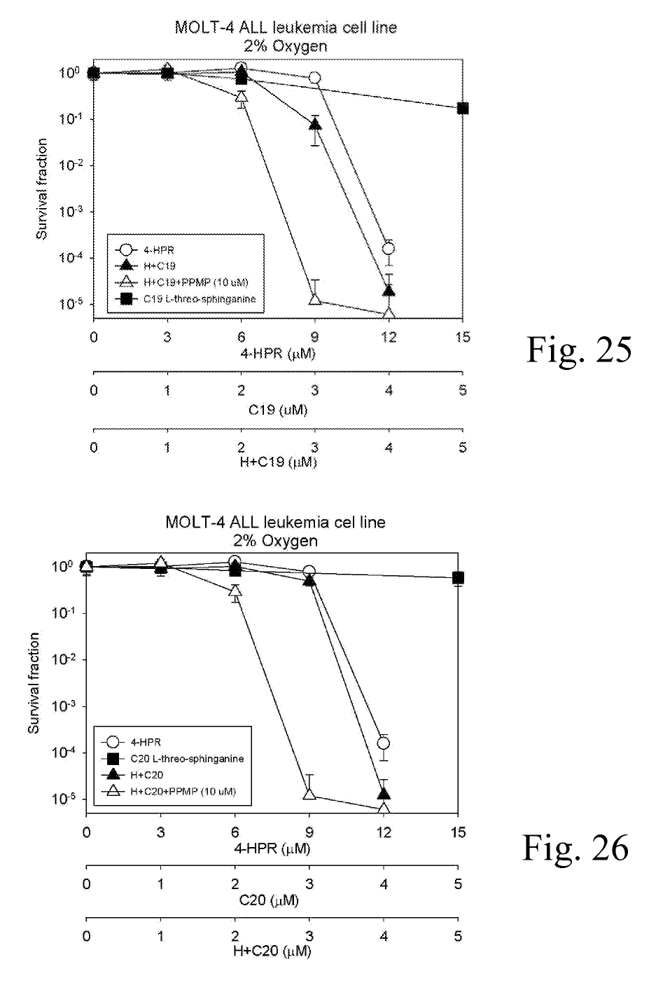

FIG. 25 is the dose-response of C19-L-threo-sphinganines in combination with fenretinide and D-threo-PPMP. Assayed by OimScan methodology at +96 hrs. Three drug combination used a fixed, minimally-toxic, concentration ofO-threo-PPMP (10 uM), an inhibitor of glucosylceramide synthase and sphingomyelin synthase. PPMP synergistically increased cytotoxicity (C.I.<1) of fenretinide +L-threo-sphinganine at most doses in the MOLT -4 ALL leukemia cell line in 2% oxygen by Combination Index Analysis Method of Chou, et al.;

FIG. 26 is the dose-response of C20-L-threo-sphinganines in combination with fenretinide and D-threo-PPMP. Assayed by DimScan methodology at +96 hrs. Three drug combination used a fixed, minimally-toxic, concentration of D-threo-PPMP (10 uM), an inhibitor of glucosylceramide synthase and sphingomyelin synthase. PPMP synergistically increased cytotoxicity (C.I.<1) of fenretinide +L-threo-sphinganine at most doses in the MOLT-4 ALL leukemia cell line in 2% oxygen by Combination Index Analysis Method of Chou, et al.

### EXAMPLES B 1- B21

Cytotoxicity Assay—Once again, cytotoxicity is determined using the DIMSCAN assay system (R. Proffitt et al., Cytometry 24, 204-213 (1996); T. Frgala et al., Mol Cancer Ther. 6:886-97, 2007). The system employs digital imaging microscopy to quantify viable cells, which selectively accumulate fluorescein diacetate to become brightly fluorescent. The system is capable of measuring cytotoxicity over a 4-5 log dynamic range by quenching the residual fluorescence of dead and dying cells with eosin Y and quantifying the total fluorescence of viable cells using digital thresholding. Measured fluorescence is directly proportionate to the number of viable cells. A comparison of the total fluorescence of a drug-treated cell population to the fluorescence of a similar number of untreated cells yields a survival fraction. In brief, 2000 to 10,000 cells/well (depending on size and growth rates) are replicate plated into 60 wells of a 96-well tissue culture plate in 0.1 mL of whole medium and allowed to attach overnight. Drug(s) are then added in 0.05 mL of whole medium to the final concentrations indicated. Drugs (fenretinide, and various chain length L-threo-sphinganines) were tested both as single agents and in a 3:1 ratio of fenretinide:sphinganine. C 18-L-threo-sphinganine, or “safingol,” was also tested and results shown for reference. There are 12 wells treated per drug concentration. Twelve wells receive drug vehicle-only to the appropriate final concentration and serve as controls for the plate. Cells are incubated for 96 hours at 37° C. in ambient air with 5% CO₂. Fluorescein diacetate is then added to each well in 0.05 mL media to a final concentration of 8 microgram/mL. Cells are incubated for a further 15 minutes at 37° C. and 0.03 mL of 0.5% eosin Y is added to each well. Total fluorescence of viable cells is then measured by digital imaging microscopy. Results were graphed as the ratio of the fluoresecence of drug-treated wells to nondrug treated control wells (i.e., Survival Fraction). All tests were performed at least twice. Representative results are shown.

Examples also shows tables containing the Combination Index (CI) calculated using CalcuSyn version 2.0 software, BIOSOFT®, Cambridge, UK, (Chou-Talalay “dose-effect” analysis, Trends Pharmacal. Sci. 4, 450-454, 1983, Chou, Cancer Res; 70:440-446, 201 0) as a measure of drug synergy for the L-threo-sphinganines tested with fenretinide. The Combination Index (CI) is a term to describe mathematical modeling of the pharmacologic effects on cytotoxicity (cell death) of two drugs in combination based. Cytotoxic “synergy” is defined as a cell death affect which is greater than would be expected from a simple product of the single agent cytotoxicities alone (i.e. an “additive” effect). By Chou-Talalay analysis, a CI<0.9 on a Fraction Affected (“Fa”, i.e. fraction of cells killed) indicates synergy, with smaller numbers indicating greater synergy; a CI of 0.9-1.1 signifies an additive or near-additive effect; and a CI>1.1 means the drug combination is antagonistic.

It is notable that multi-log cytotoxicity was achieved in cell lines that were p53 null or mutant and in cell lines that are highly resistant to alkylating agents.

Results demonstrate that, while not all are equally active in all human cancer cell lines, all non-C 18-L-threo-sphinganines can increase fenretinide cytotoxicity, either additively or synergistically, in a broad range of human cancer cell types, including both solid and hematopoietic cancers, and adult and pediatric cancers.

### Example B1

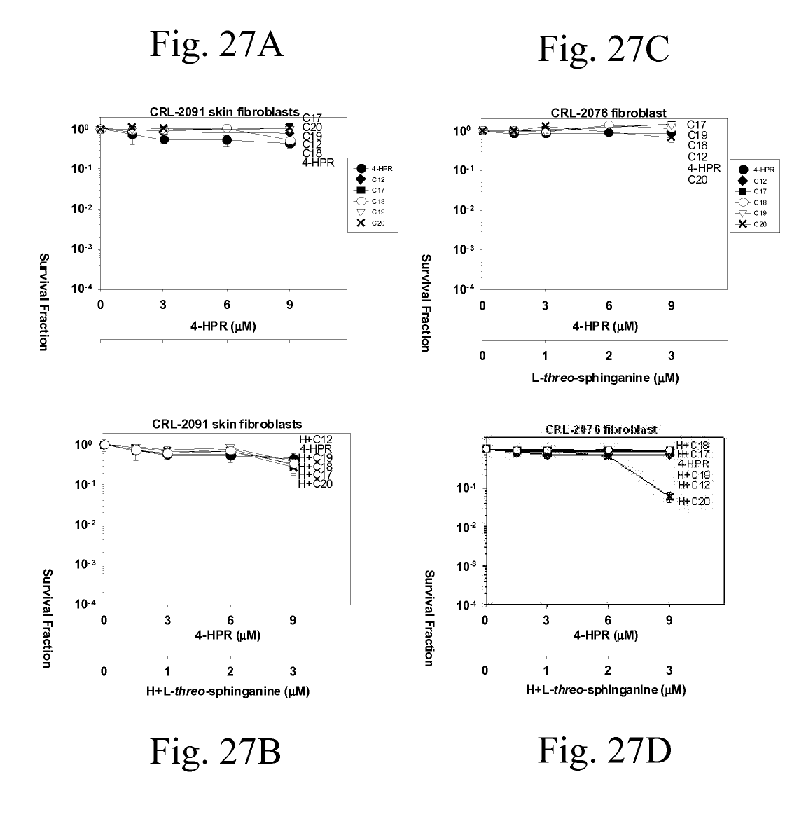

Fenretinide and L-threo-sphinganines tested in normal human fibroblast (normal skin cell) cell lines, CRL-2091 and CRL-2076. Results- show that L-threo-sphinganines and fenretinide combinations are minimally cytotoxic in normal human cell. Reference is made to the graphs plotting the results shown in FIGS. 27 A - 27D which results are reported in TABLES 1.1 and 1.2 below.

TABLE 1.1
CRL-2091, 20% O2

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |

| | | | | | | | | | | | |
|---|---|---|---|---|---|---|---|---|---|---|---|
| 1.5 | 0.5 | 0.27 | >1 | 0.32 | >1 | 0.26 | >1 | 0.13 | >1 | 0.16 | >1 |
| 3 | 1 | 0.44 | 0.76 | 0.40 | >1 | 0.39 | >1 | 0.25 | >1 | 0.29 | >1 |
| 6 | 2 | 0.42 | >1 | 0.21 | >1 | 0.29 | >1 | 0.13 | >1 | 0.29 | >1 |
| 9 | 3 | 0.54 | >1 | 0.69 | >1 | 0.58 | >1 | 0.62 | >1 | 0.73 | 0.33 |

TABLE 1.2
CRL-2076, 20% O₂

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.17 | 1 | | | | | 0.10 | >1 | 0.08 | 1 | 0.05 | >1 | 0.20 | >1 | | |
| 3 | 1 | 0.30 | >1 | | | | | 0.10 | >1 | 0.09 | >1 | 0.04 | >1 | 0.11 | 1 | | |
| 6 | 2 | 0.29 | >1 | | | | | 0.12 | >1 | 0.06 | >1 | 0.09 | >1 | 0.35 | >1 | | |
| 9 | 3 | 0.30 | >1 | | | | | 0.08 | >1 | 0.07 | >1 | 0.15 | >1 | 0.94 | >1 | | |

### Example B2

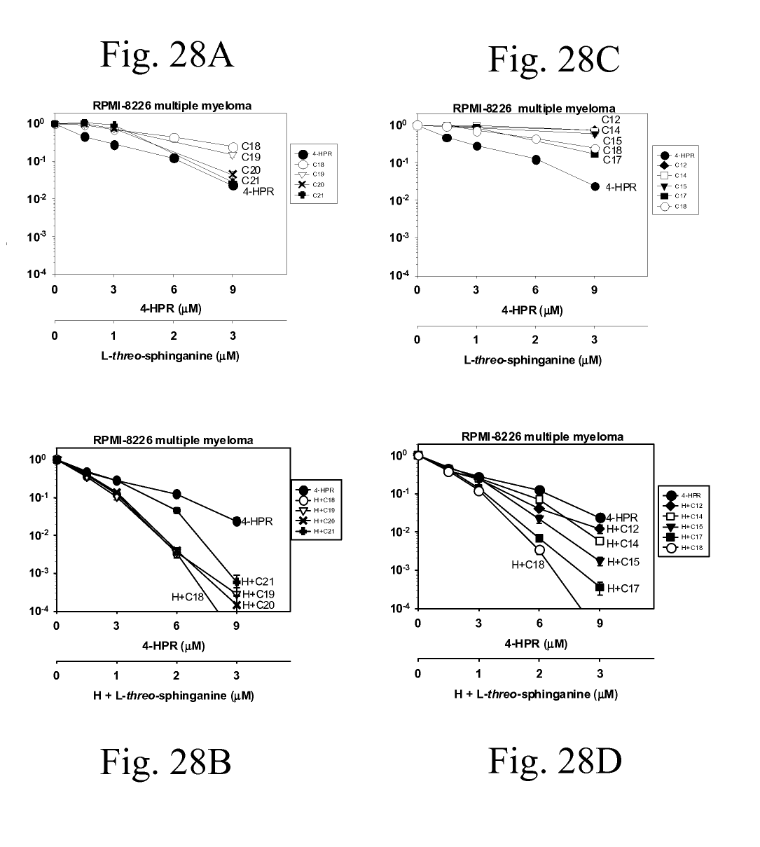

Fenretinide and L-threo-sphinganines tested in human Multiple Myeloma (a cancer of the blood and bone marrow) cell line, RPMI-8226. Results show that at least some L-threo-sphinganines increased fenretinide cytotoxicity, additively or synergistically, depending on the drug concentrations tested. Reference is made to the graphs plotting the results shown in FIGS. 28A-28D which results are reported in TABLE 2.1 below.

TABLE 2.1
RPMI-8226

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.6 | 0.8 | 0.606 | 0.8 | 0.518 | 1.0 | 0.549 | 1.12 | 0.616 | 1.0 | 0.649 | 0.9 | 0.583 | 1.0 | 0.517 | 1.2 |
| 3 | 1 | 0.757 | 1.0 | 0.747 | 1.1 | 0.75 | 1.1 | 0.859 | 1.00 | 0.879 | 0.9 | 0.895 | 0.9 | 0.867 | 1.1 | 0.722 | 1.6 |
| 6 | 2 | 0.95 | 0.7 | 0.93 | 1.0 | 0.978 | 0.5 | 0.993 | 0.43 | 0.997 | 0.2 | 0.996 | 0.5 | 0.996 | 0.9 | 0.955 | 1.6 |
| 9 | 3 | 0.988 | 0.5 | 0.994 | 0.4 | 0.998 | 0.2 | 1.000 | >0.1 | 1 | >0.1 | 1.000 | >0.1 | 1 | 0.3 | 0.999 | 1.1 |

### Example B3

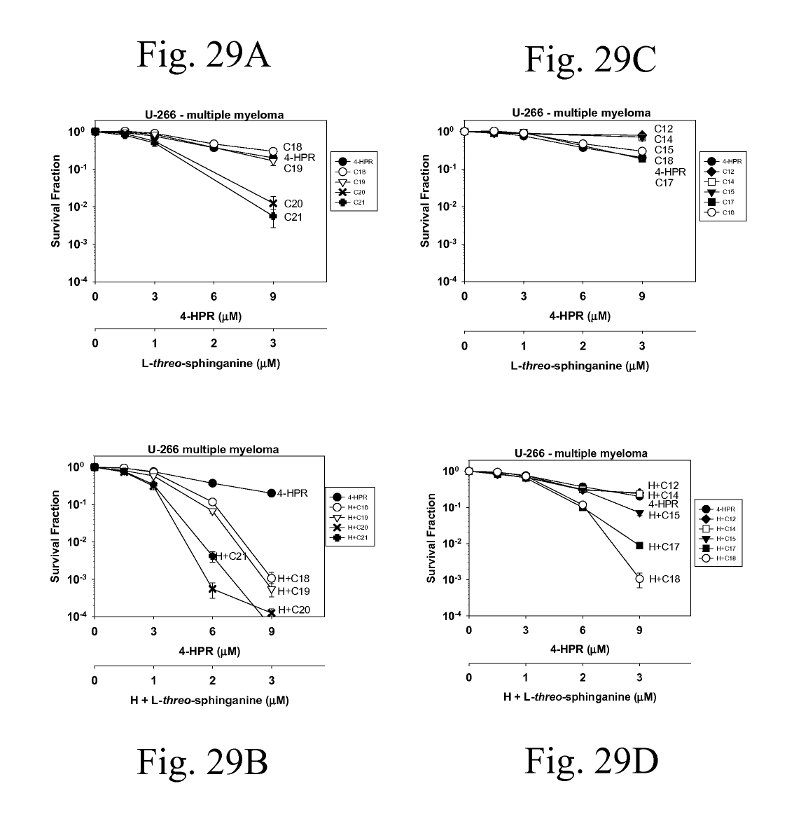

Fenretinide and L-threo-sphinganines tested in human Multiple Myeloma (a cancer of the blood and bone marrow) cell line, U-266. Results show that at least some L-threo-sphinganines increased fenretinide cytotoxicity, additively or synergistically, depending on the drug concentrations tested. Reference is made to the graphs plotting the results shown in FIGS. 29A-29D which results are reported in TABLE 3.1 below.

TABLE 3.1
U-266

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.176 | 0.8 | 0.163 | 0.9 | 0.085 | 1.2 | 0.134 | 1.3 | 0.048 | 1.4 | 0.19 | 1.0 | 0.259 | 1.2 | 0.225 | 1.4 |
| 3 | 1 | 0.326 | 0.9 | 0.315 | 1.1 | 0.241 | 1.5 | 0.35 | 1.5 | 0.268 | 1.5 | 0.414 | 1.4 | 0.688 | 1.3 | 0.658 | 1.5 |
| 6 | 2 | 0.697 | 0.9 | 0.687 | 1.0 | 0.697 | 1.2 | 0.901 | 0.9 | 0.883 | 1.3 | 0.933 | 0.9 | 0.999 | 0.4 | 0.996 | 0.6 |
| 9 | 3 | 0.748 | 1.2 | 0.76 | 1.3 | 0.929 | 0.9 | 0.991 | 0.4 | 0.999 | 0.8 | 0.999 | 0.3 | 1.000 | >0.1 | 1 | >0.1 |

### Example B4

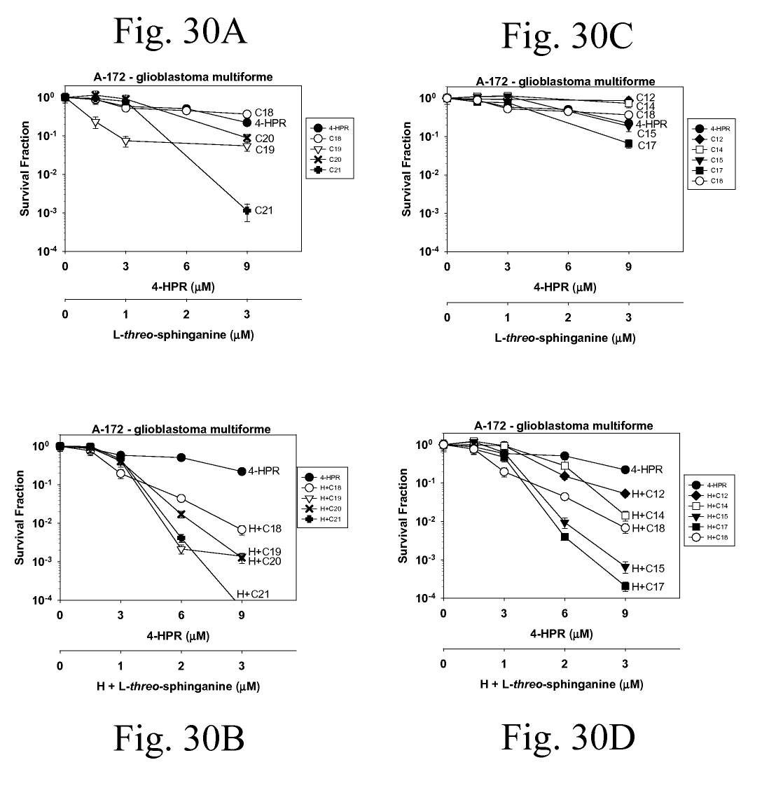

Fenretinide and L-threo-sphinganines tested in human Glioblastoma multiforme (brain cancer) cell line, A-172. Results show that at least some L-threo-sphinganines increased fenretinide cytotoxicity, additively or synergistically, depending on the drug concentrations tested. Reference is made to the graphs plotting the results shown in FIGS. 30A-30D which results are reported in TABLE 4.1 below.

TABLE 4.1
A-172

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.024 | 6.1 | 0.001 | 23.7 | 0.001 | 23.7 | 0.232 | 1.4 | 0.233 | 1.3 | 0.086 | 84.4 | 0.039 | 2.8 | 0.006 | 8.8 |
| 3 | 1 | 0.065 | 4.4 | 0.065 | 3.8 | 0.378 | 1.4 | 0.529 | 1.4 | 0.803 | 0.5 | 0.578 | 8.1 | 0.594 | 1.0 | 0.537 | 1.6 |
| 6 | 2 | 0.849 | 0.4 | 0.717 | 1.0 | 0.991 | 0.4 | 0.996 | 0.2 | 0.956 | 0.3 | 0.998 | >0.1 | 0.983 | 0.6 | 0.996 | 0.8 |
| 9 | 3 | 0.981 | 0.2 | 0.958 | 0.5 | 0.998 | 0.4 | 1.000 | >0.1 | 0.993 | 0.1 | 1.000 | >0.1 | 1.000 | >0.1 | 1.000 | >0.1 |

### Example B5

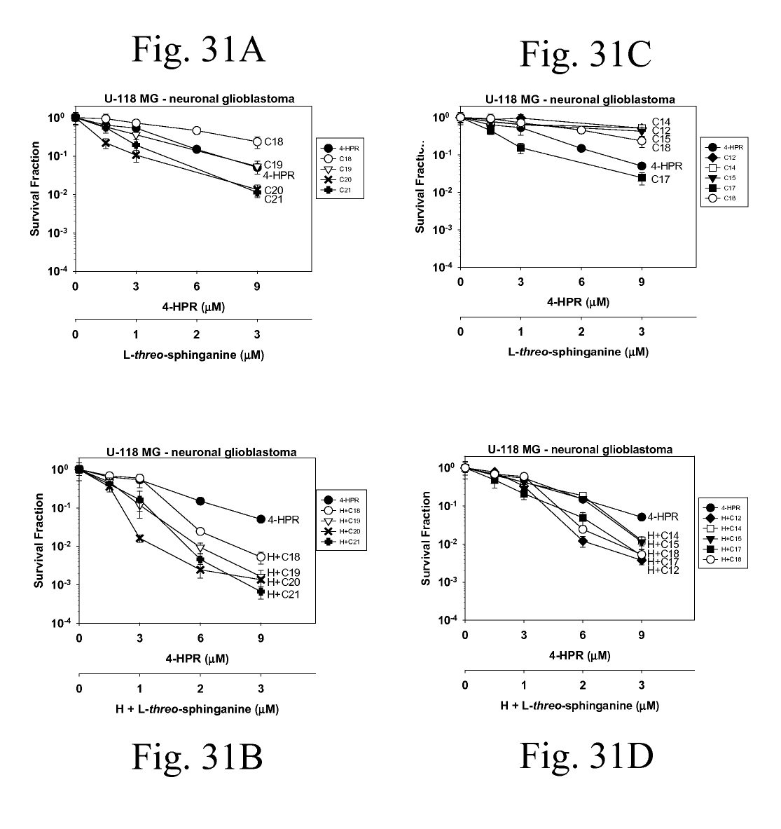

Fenretinide and L-threo-sphinganines tested in human Glioblastoma (brain cancer) cell line; U-118. Results show that at least some L-threo-sphinganines increased fenretinide cytotoxicity, additively or synergistically, depending on the drug concentrations tested. Reference is made to the graphs plotting the results shown in FIGS. 31A-31D which results are reported in TABLE 5.1 below.

TABLE 5.1
U-118 MG

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.226 | 1.5 | 0.318 | 1.4 | 0.371 | 0.4 | 0.519 | 1.7 | 0.317 | 1.4 | 0.530 | 1.3 | 0.645 | 1.8 | 0.588 | 1.3 |
| 3 | 1 | 0.671 | 1.0 | 0.586 | 1.2 | 0.577 | 0.5 | 0.791 | 1.8 | 0.407 | 2.3 | 0.882 | 0.9 | 0.984 | 0.5 | 0.836 | 1.5 |
| 6 | 2 | 0.988 | 0.3 | 0.814 | 1.2 | 0.847 | 0.4 | 0.951 | 1.5 | 0.976 | 0.5 | 0.991 | 0.4 | 0.998 | 0.3 | 0.995 | 0.6 |
| 9 | 3 | 0.996 | 0.2 | 0.988 | 0.4 | 0.989 | 0.1 | 0.995 | 0.7 | 0.995 | 0.3 | 0.998 | 0.3 | 0.999 | 0.3 | 0.999 | 0.5 |

### Example B6

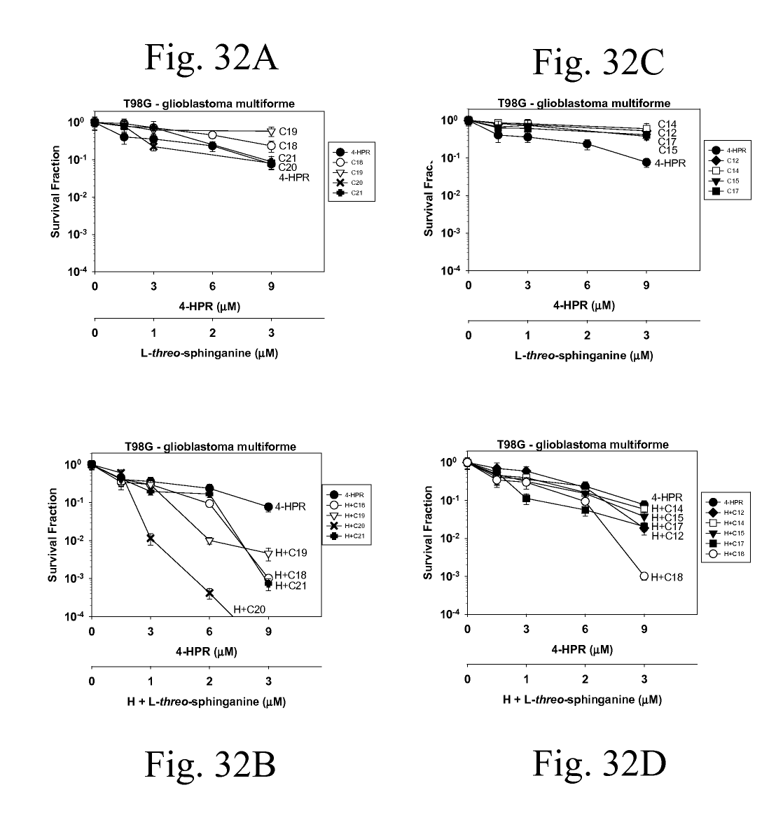

Fenretinide and L-threo-sphinganines tested in human Glioblastoma multiforme (brain cancer) cell line, T98G. Results show that at least some L-threo-sphinganines increased fenretinide cytotoxicity, additively or synergistically, depending on the drug concentrations tested. Reference is made to the graphs plotting the results shown in FIGS. 32A-32D which results are reported in TABLE 6.1 below.

TABLE 6.1
T98G

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.306 | 2.8 | 0.544 | 1.0 | 0.516 | 1.3 | 0.447 | 1.7 | 0.657 | 0.786 | 0.584 | 0.9 | 0.397 | 2.4 | 0.574 | 1.2 |
| 3 | 1 | 0.408 | 3.6 | 0.602 | 1.6 | 0.683 | 1.3 | 0.887 | 0.3 | 0.701 | 1.316 | 0.704 | 1.1 | 0.989 | 0.2 | 0.805 | 1.0 |
| 6 | 2 | 0.781 | 1.5 | 0.839 | 1.0 | 0.849 | 1.0 | 0.944 | 0.3 | 0.906 | 0.767 | 0.990 | 0.1 | 1.000 | >0.1 | 0.832 | 1.8 |
| 9 | 3 | 0.981 | 0.2 | 0.941 | 0.5 | 0.962 | 0.4 | 0.980 | 0.2 | 0.999 | 0.023 | 0.995 | 0.1 | 1.000 | >0.1 | 0.999 | 0.1 |

### Example B7

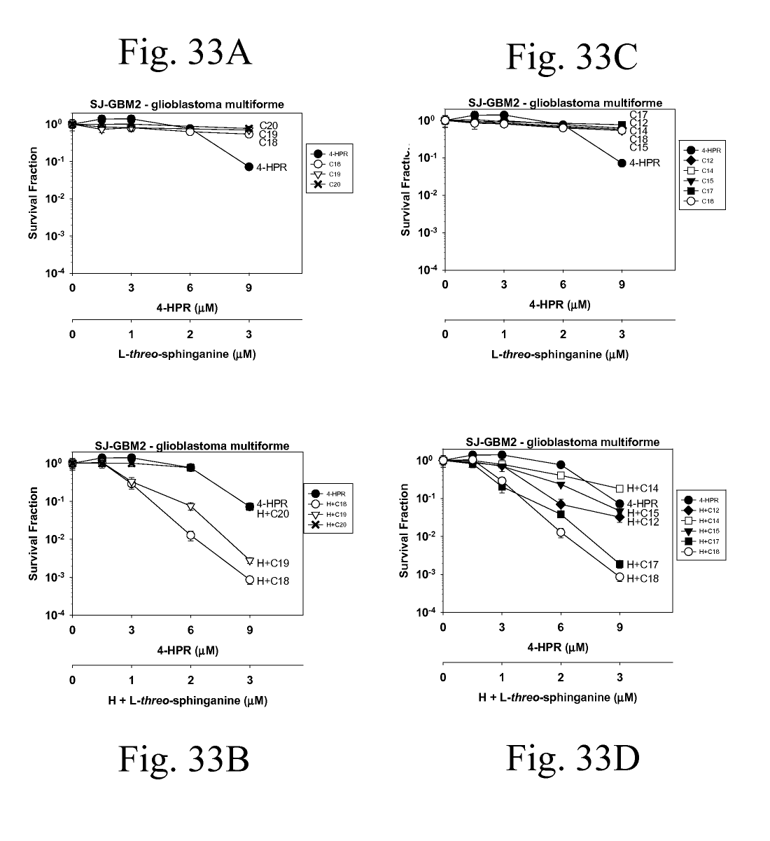

Fenretinide and L-threo-sphinganines tested in human Glioblastoma multiforme (brain cancer) cell line, SJ-GBM2. Results show that at least some L-threo-sphinganines increased fenretinide cytotoxicity, additively or synergistically, depending on the drug concentrations tested. Reference is made to the graphs plotting the results shown in FIGS. 33A-33D which results are reported in TABLE 7.1 below.

TABLE 7.1
SJ-GBM2

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.133 | 0.5 | 0.007 | 5.0 | 0.086 | 1.7 | 0.188 | 0.5 | 0.001 | >>1 | 0.001 | >>1 | 0.055 | 0.5 | 0.131 | 1.4 |
| 3 | 1 | 0.271 | 0.7 | 0.209 | 1.2 | 0.301 | 1.1 | 0.796 | 0.5 | 0.712 | 0.5 | 0.672 | 0.4 | 0.737 | 0.6 | 0.470 | 1.9 |
| 6 | 2 | 0.930 | 0.7 | 0.598 | 1.2 | 0.765 | 0.9 | 0.962 | 0.7 | 0.987 | 0.6 | 0.925 | 0.7 | 0.998 | 0.7 | 0.946 | 2.1 |
| 9 | 3 | 0.968 | 0.9 | 0.820 | 1.4 | 0.953 | 1.0 | 0.998 | 0.7 | 0.999 | 0.7 | 0.997 | 0.8 | 1.000 | 0.3 | 0.996 | 2.0 |

### Example B8

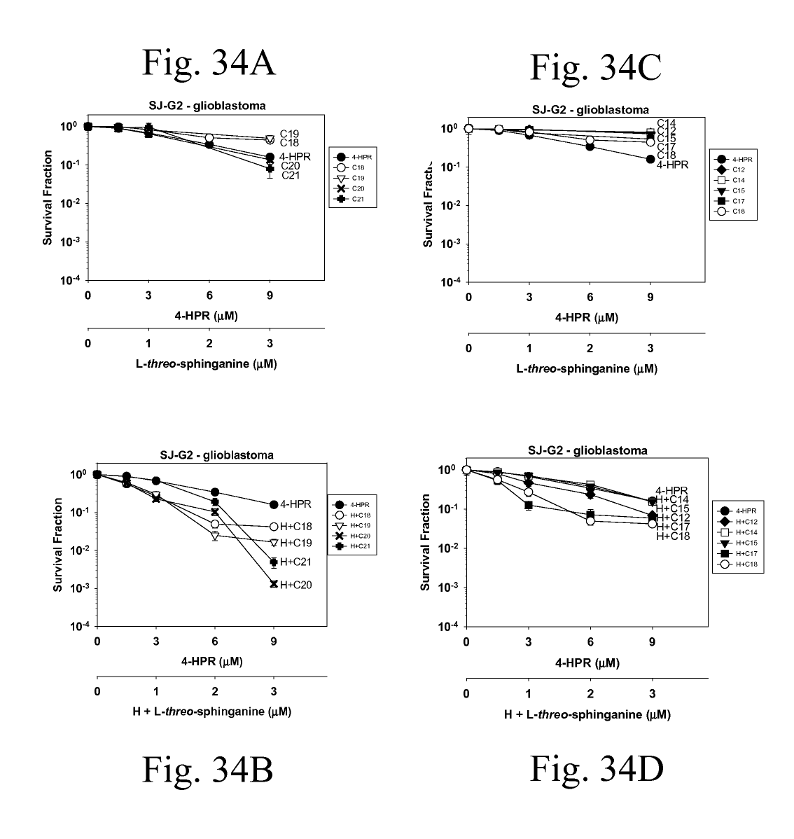

Fenretinide and L-threo-sphinganines tested in human Glioblastoma (brain cancer) cell line, SJ-G2. Results show that at least some L-threo-sphinganines increased fenretinide cytotoxicity, additively or synergistically, depending on the drug concentrations tested. Reference is made to the graphs plotting the results shown in FIGS. 34A-34D which results are reported in TABLE 8.1 below.

TABLE 8.1
SJ-G2

| | L-threo- | | | | | | | | | | | | | | | | |
|---|---|---|---|---|---|---|---|---|---|---|---|---|---|---|---|---|---|
| 4-HPR | Sphinganiine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.199 | 0.8 | 0.103 | 1.3 | 0.135 | 1.1 | 0.452 | 0.6 | 0.437 | 0.7 | 0.389 | 0.7 | 0.408 | 0.9 | 0.118 | 1.5 |
| 3 | 1 | 0.530 | 0.7 | 0.307 | 1.3 | 0.282 | 1.3 | 0.874 | 0.4 | 0.728 | 0.7 | 0.700 | 0.8 | 0.773 | 0.8 | 0.299 | 1.9 |
| 6 | 2 | 0.764 | 0.8 | 0.575 | 1.4 | 0.620 | 1.3 | 0.928 | 0.6 | 0.950 | 0.5 | 0.975 | 0.6 | 0.896 | 1.1 | 0.809 | 1.5 |
| 9 | 3 | 0.931 | 0.6 | 0.845 | 1.0 | 0.846 | 1.0 | 0.941 | 0.8 | 0.958 | 0.6 | 0.983 | 0.7 | 0.999 | 0.2 | 0.995 | 0.5 |

### Example B9

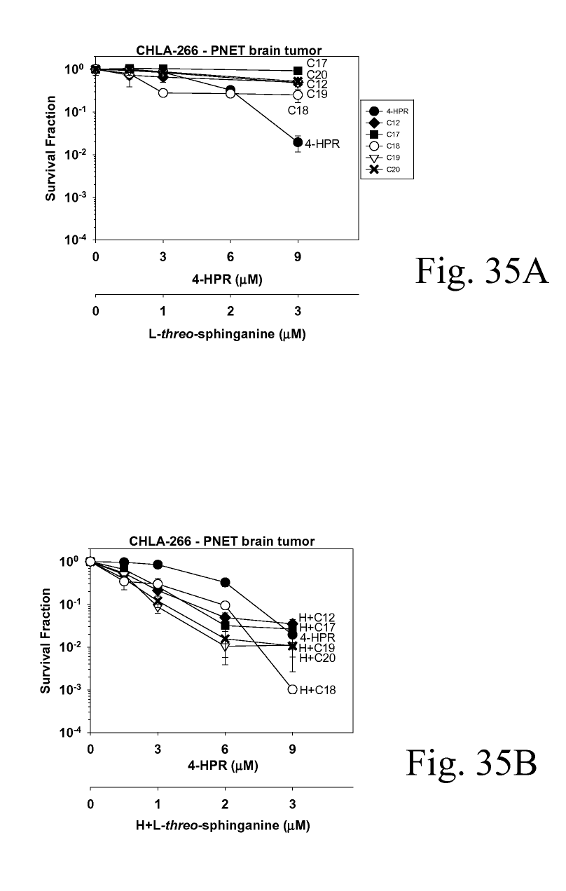

Fenretinide and L-threo-sphinganines tested in human primitive neuroectodermal tumor (PNET) (brain cancer) cell line, CHLA-266. Results show that at least some L-threo-sphinganines increased fenretinide cytotoxicity, additively or synergistically, depending on the drug concentrations tested. Reference is made to the graphs plotting the results shown in FIGS. 35A & 35B which results are reported in TABLE 9.1 below.

TABLE 9.1
CHLA-266

| | L-threo- | | | | | | | | | | | | | | | | |
|---|---|---|---|---|---|---|---|---|---|---|---|---|---|---|---|---|---|
| 4-HPR | Sphinganine | C12 | | C14 | | C15 | | C17 | | C18 | | C19 | | C20 | | C21 | |
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |

| | | | | | | | | | | | |
|---|---|---|---|---|---|---|---|---|---|---|---|
| 1.5 | 0.5 | 0.464 | 0.6 | 0.342 | 0.6 | 0.657 | 0.5 | 0.502 | 0.5 | 0.604 | 0.5 |
| 3 | 1 | 0.788 | 0.6 | 0.739 | 0.9 | 0.701 | 1.0 | 0.913 | 0.5 | 0.880 | 0.8 |
| 6 | 2 | 0.951 | 0.7 | 0.968 | 1.1 | 0.906 | 1.2 | 0.990 | 0.5 | 0.984 | 1.1 |
| 9 | 3 | 0.965 | 1.0 | 0.974 | 1.6 | 0.999 | 0.4 | 0.989 | 0.7 | 0.989 | 1.5 |

### Example B10

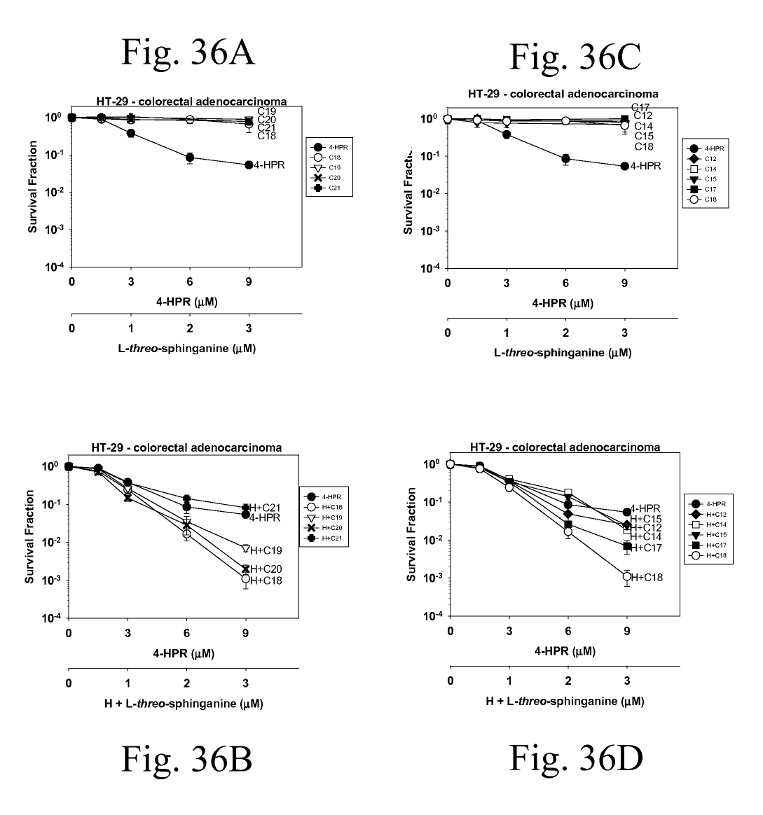

Fenretinide and L-threo-sphinganines tested in human colorectal adenocarcinoma (colon cancer) cell line, HT-29. Results show that at least some L-threo-sphinganines increased fenretinide cytotoxicity, additively or synergistically, depending on the drug concentrations tested. Reference is made to the graphs plotting the results shown in FIGS. 36A-36D which results are reported in TABLE 10.1 below.

TABLE 10.1
HT-29

| 4-HPR | L-threo-Sphinganine | C12 | | C14 | | C15 | | C17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.217 | 1.0 | 0.215 | 1.0 | 0.204 | 2.0 | 0.112 | 1.1 | 0.243 | 0.9 | 0.138 | 1.2 | 0.287 | 0.8 | 0.149 | 1.2 |
| 3 | 1 | 0.642 | 1.2 | 0.598 | 1.3 | 0.660 | 0.8 | 0.661 | 0.8 | 0.756 | 0.7 | 0.736 | 1.0 | 0.852 | 0.6 | 0.630 | 1.2 |
| 6 | 2 | 0.951 | 1.3 | 0.822 | 1.8 | 0.859 | 1.1 | 0.974 | 0.6 | 0.983 | 0.5 | 0.965 | 0.8 | 0.971 | 0.6 | 0.857 | 1.8 |
| 9 | 3 | 0.975 | 1.6 | 0.982 | 1.4 | 0.976 | 0.8 | 0.993 | 0.5 | 0.999 | 0.3 | 0.993 | 0.8 | 0.998 | 0.4 | 0.918 | 2.3 |

### Example B11

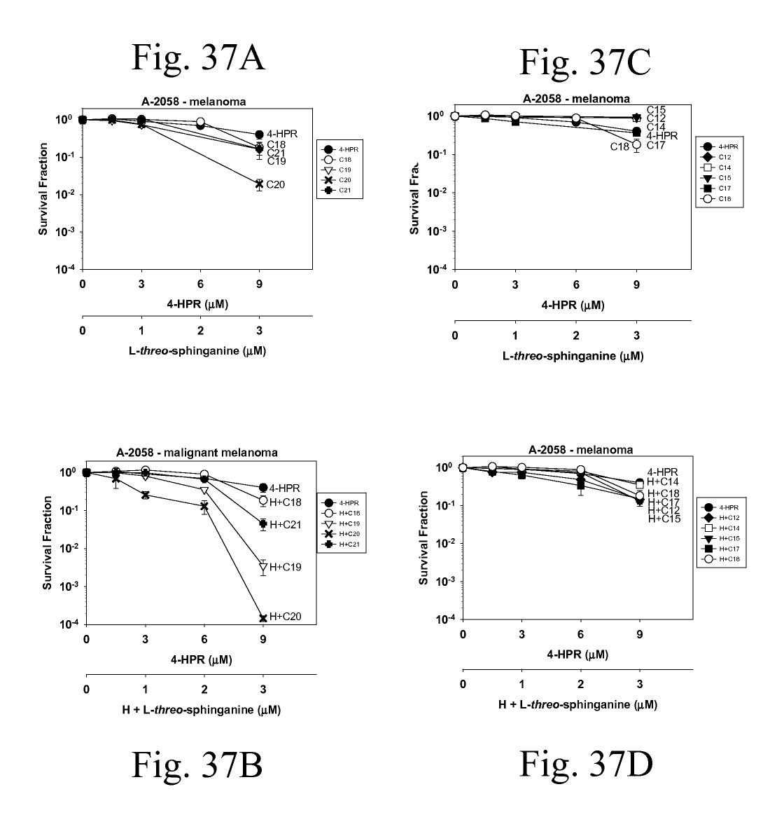

Fenretinide and L-threo-sphinganines tested in human melanoma (skin cancer) cell line, A-2058. Results show that at least some L-threo-sphinganines increased fenretinide cytotoxicity, additively or synergistically, depending on the drug concentrations tested. Reference is made to the graphs plotting the results shown in FIGS. 37A-37D which results are reported in TABLE 11.1 below.

TABLE 11.1
A-2058

| 4-HPR | L-threo-Sphinganine | C12 | | C14 | | C15 | | FₐC17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | | Cl | Fₐ | Cl | Fₐ | Cl | Fₐ | Cl | Fₐ | Cl |
| 1.5 | 0.5 | 0.244 | 0.3 | 0.045 | 1.1 | 0.053 | 0.5 | 0.211 | 0.9 | 0.001 | >1 | 0.016 | 2.1 | 0.311 | 0.7 | 0.016 | 0.7 |
| 3 | 1 | 0.247 | 0.6 | 0.088 | 1.2 | 0.106 | 0.9 | 0.359 | 1.2 | 0.01 | >1 | 0.198 | 1.7 | 0.742 | 1.0 | 0.016 | 1.4 |
| 6 | 2 | 0.518 | 0.9 | 0.193 | 1.4 | 0.262 | 1.6 | 0.664 | 1.4 | 0.099 | >1 | 0.648 | 1.8 | 0.870 | 1.7 | 0.333 | 1.9 |
| 9 | 3 | 0.854 | 1.0 | 0.650 | 1.3 | 0.867 | 1.7 | 0.849 | 1.5 | 0.815 | 1.22 | 0.997 | 0.8 | 1.000 | 0.5 | 0.955 | 1.9 |

### Example B12

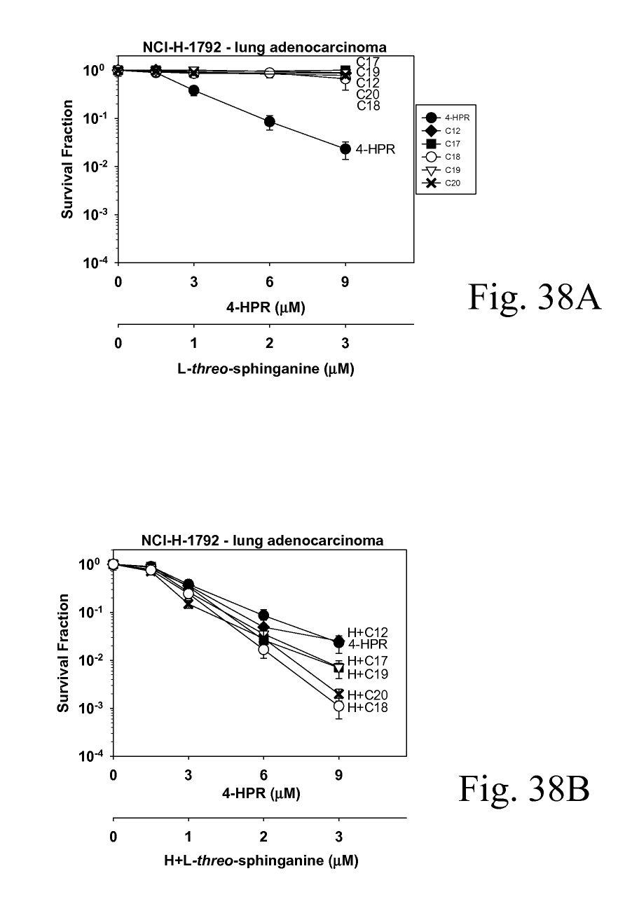

Fenretinide and L-threo-sphinganines tested in human lung adenocarcinoma (lung cancer) cell line, NCI-H-1792. Results show that at least some L-threo-sphinganines increased fenretinide cytotoxicity, additively or synergistically, depending on the drug concentrations tested. Reference is made to the graphs plotting the results shown in FIGS. 38A-38B which results are reported in TABLE 12.1 below.

TABLE 12.1
NCI-H-1792

| 4-HPR | L-threo-Sphinganine | C12 | | C14 | | C15 | | C17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |

| | | | | | | | | | | | |
|---|---|---|---|---|---|---|---|---|---|---|---|
| 1.5 | 0.5 | 0.217 | 1.0 | 0.112 | 1.1 | 0.243 | 1.2 | 0.138 | 1.1 | 0.287 | 0.9 |
| 3 | 1 | 0.542 | 1.3 | 0.661 | 1.0 | 0.756 | 0.8 | 0.736 | 1.1 | 0.852 | 0.7 |
| 6 | 2 | 0.951 | 1.4 | 0.974 | 0.9 | 0.983 | 0.6 | 0.965 | 1.2 | 0.972 | 0.7 |
| 9 | 3 | 0.975 | 1.8 | 0.993 | 1.0 | 0.999 | 0.4 | 0.993 | 0.9 | 0.998 | 0.5 |

### Example B13

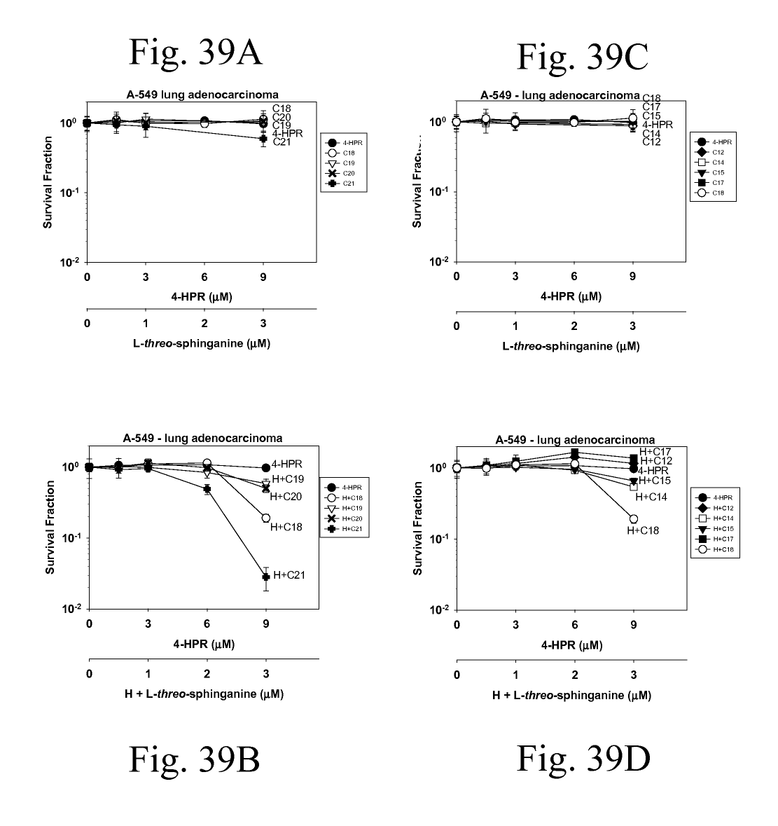

Fenretinide and L-threo-sphinganines tested in human lung adenocarcinoma (lung cancer) cell line, A-549. Results show that at least some L-threo-sphinganines increased fenretinide cytotoxicity, additively or synergistically, depending on the drug concentrations tested. Reference is made to the graphs plotting the results shown in FIGS. 39A-39D which results are reported in TABLE 13.1 below.

TABLE 13.1
A-549

| 4-HPR | L-threo-Sphinganine | C12 | | C14 | | C15 | | C17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.01 | 2.5 | 0.01 | 1 | 0.000 | 1.9 | 0.01 | 1.6 | 0.012 | 1.0 | 0.000 | 1.6 | 0.000 | 2.1 | 0.078 | 0.9 |
| 3 | 1 | 0.010 | 1.0 | 0.02 | 1.0 | 0.000 | 2.1 | 0.010 | 1.5 | 0.05 | 1.0 | 0.007 | 0.6 | 0.000 | 4.1 | 0.044 | 2.5 |
| 6 | 2 | 0.010 | 1.0 | 0.072 | 1.1 | 0.044 | 0.9 | 0.010 | 1.0 | 0.200 | 1.0 | 0.159 | 0.7 | 0.017 | 1.1 | 0.51 | 0.8 |
| 9 | 3 | 0.020 | 1.0 | 0.461 | 0.5 | 0.342 | 0.8 | 0.010 | 1.1 | 0.700 | 0.6 | 0.413 | 0.9 | 0.499 | 1.1 | 0.972 | 0.4 |

### Example B14

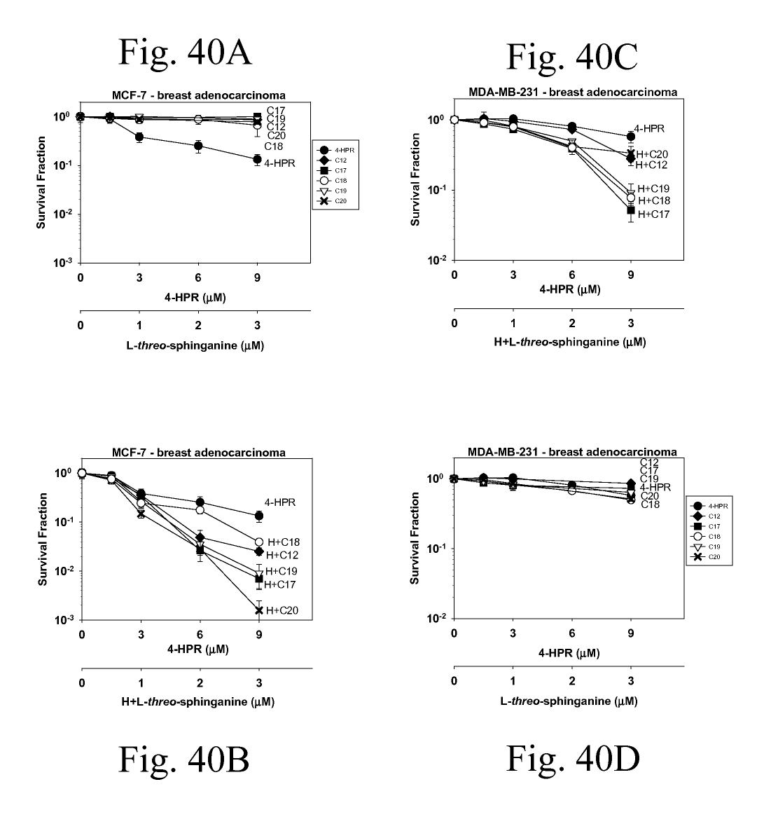

Fenretinide and L-threo-sphinganines tested in human breast adenocarcinoma (breast cancer) cell lines, MCF-7 and MDA-MB-231. Results show that at least some L-threo-sphinganines increased fenretinide cytotoxicity, additively or synergistically, depending on the drug concentrations tested. Reference is made to the graphs plotting the results shown in FIGS. 40A-40D which results are reported in TABLES 14.1 and 14.2 below.

TABLE 14.1
MCF-7

| 4-HPR | L-threo-Sphinganine | C12 | | C14 | | C15 | | C17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |

| | | | | | | | | | | | |
|---|---|---|---|---|---|---|---|---|---|---|---|
| 1.5 | 0.5 | 0.217 | 1.0 | 0.112 | 1.3 | 0.243 | 1.2 | 0.138 | 1.2 | 0.287 | 0.8 |
| 3 | 1 | 0.642 | 1.0 | 0.661 | 0.7 | 0.758 | 0.6 | 0.736 | 0.8 | 0.852 | 0.4 |
| 6 | 2 | 0.951 | 0.9 | 0.974 | 0.4 | 0.824 | 0.9 | 0.965 | 0.8 | 0.971 | 0.3 |
| 9 | 3 | 0.975 | 1.2 | 0.993 | 0.4 | 0.961 | 0.6 | 0.991 | 0.9 | 0.998 | 0.1 |

TABLE 14.2
MDA-MB-231.20% O2

| 4-HPR | L-threo-Sphinganine | C12 | | C14 | | C15 | | C17 | | C18 | | C19 | | C20 | |
|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl |

| | | | | | | | | | | | |
|---|---|---|---|---|---|---|---|---|---|---|---|
| 1.5 | 0.5 | 1E−07 | 1.5 | 0.127 | 1.3 | 0.088 | 1.8 | 0.078 | 1.2 | 0.011 | 2.0 |
| 3 | 1 | 0.052 | 0.9 | 0.265 | 0.9 | 0.207 | 1.8 | 0.195 | 1.2 | 0.190 | 1.1 |
| 6 | 2 | 0.272 | 1.6 | 0.610 | 0.8 | 0.597 | 1.3 | 0.503 | 1.1 | 0.583 | 1.3 |
| 9 | 3 | 0.720 | 2.0 | 0.948 | 0.7 | 0.922 | 0.7 | 0.909 | 0.7 | 0.664 | 1.8 |

### Example B15

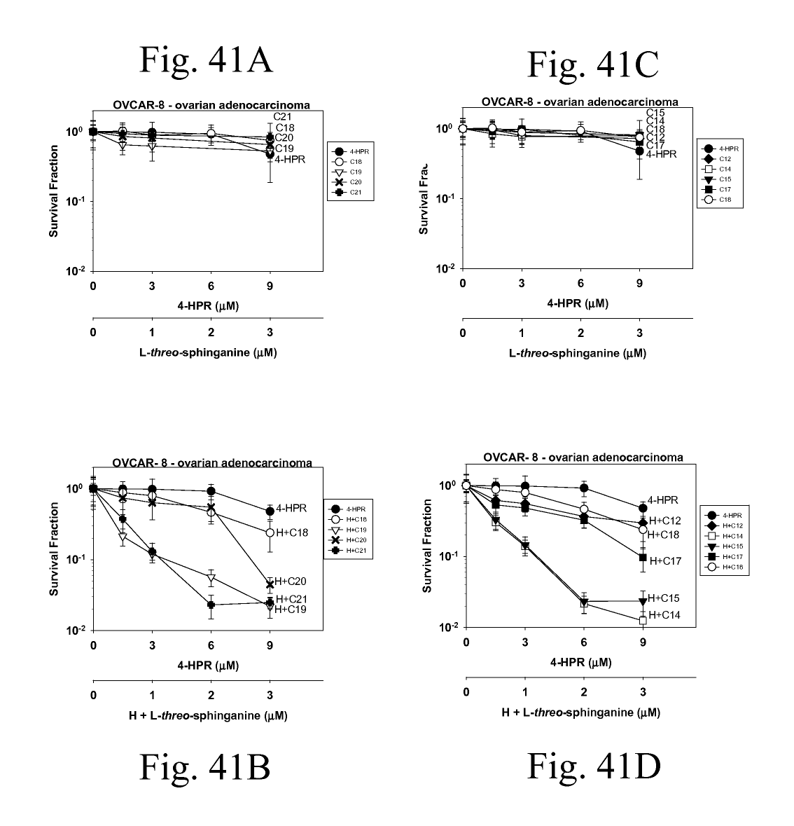

Fenretinide and L-threo-sphinganines tested in human ovarian adenocarcinoma (ovarian cancer) cell line, OVCAR-8. Results show that at least some L-threo-sphinganines increased fenretinide cytotoxicity, additively or synergistically, depending on the drug concentrations tested. Reference is made to the graphs plotting the results shown in FIGS. 41A-41D which results are reported in TABLE 15.1 below.

TABLE 15.1
OVCAR-3

| 4-HPR | L-threo-Sphinganine | C12 | | C14 | | C15 | | C17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.382 | 0.3 | 0.696 | 0.1 | 0.668 | 0.1 | 0.467 | 0.2 | 0.122 | 1.5 | 0.787 | 0.1 | 0.259 | 0.5 | 0.626 | 0.1 |
| 3 | 1 | 0.445 | 0.5 | 0.860 | 0.1 | 0.854 | 0.1 | 0.519 | 0.4 | 0.202 | 1.5 | 0.881 | 0.1 | 0.366 | 0.6 | 0.870 | 0.1 |
| 6 | 2 | 0.634 | 0.6 | 0.978 | 0.1 | 0.977 | 0.1 | 0.675 | 0.5 | 0.536 | 0.7 | 0.943 | 0.1 | 0.453 | 0.8 | 0.977 | 0.1 |
| 9 | 3 | 0.704 | 0.8 | 0.988 | 0.1 | 0.977 | 0.2 | 0.903 | 0.4 | 0.761 | 0.5 | 0.978 | 0.1 | 0.956 | 0.2 | 0.975 | 0.2 |

### Example B16

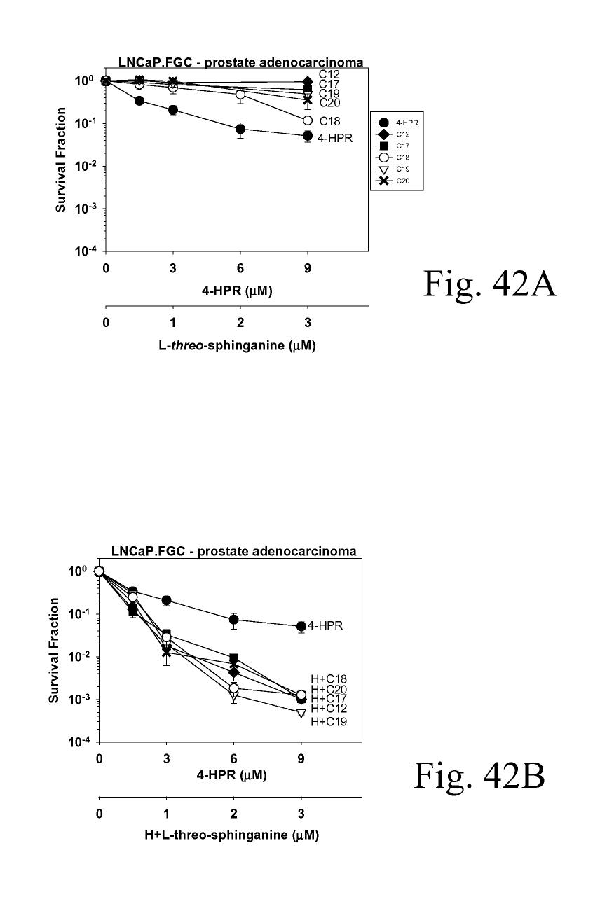

Fenretinide and L-threo-sphinganines tested in human prostate adenocarcinoma (prostate cancer) cell line, LNCaP. Results show that at least some L-threo-sphinganines increased fenretinide cytotoxicity, additively or synergistically, depending on the drug concentrations tested. Reference is made to the graphs plotting the results shown in FIGS. 42A-42B which results are reported in TABLE 16.1 below.

TABLE 16.1
LNCaP.FGC

| 4-HPR | L-threo-Sphinganine | C12 | | C14 | | C15 | | C17 | | C18 | | C19 | | C20 | |
|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl |

| | | | | | | | | | | | |
|---|---|---|---|---|---|---|---|---|---|---|---|
| 1.5 | 0.5 | 0.870 | 0.5 | 0.887 | 0.3 | 0.752 | 0.9 | 0.697 | 1.0 | 0.812 | 0.7 |
| 3 | 1 | 0.982 | 0.3 | 0.967 | 0.3 | 0.971 | 0.3 | 0.980 | 0.5 | 0.987 | 0.4 |
| 6 | 2 | 0.996 | 0.4 | 0.991 | 0.2 | 0.998 | 0.1 | 0.999 | 0.5 | 0.993 | 0.7 |
| 9 | 3 | 0.999 | 0.4 | 0.999 | 0.1 | 0.999 | 0.1 | 1.000 | 0.2 | 0.999 | 0.8 |

### Example B17

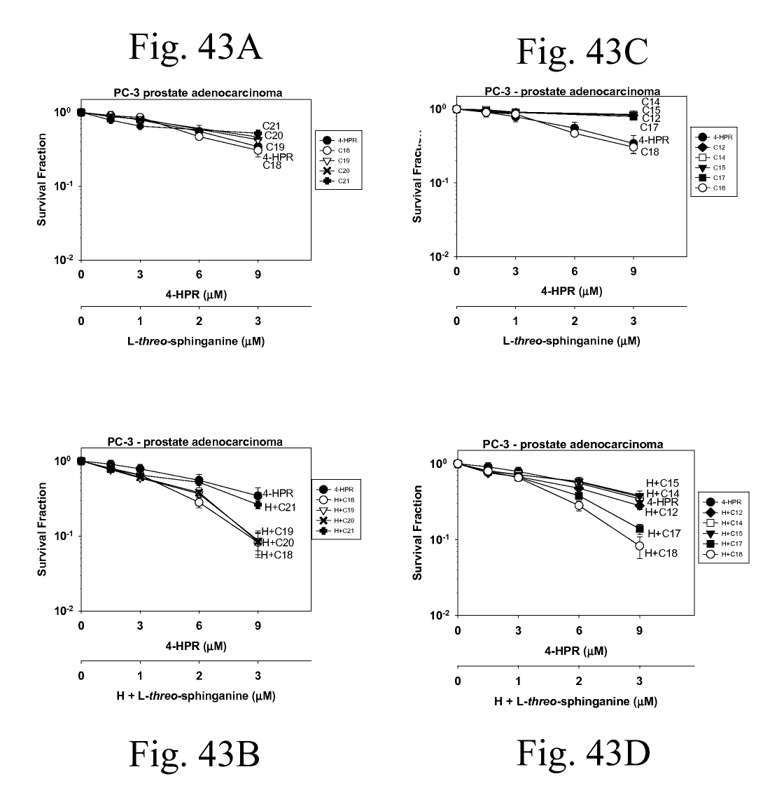

Fenretinide and L-threo-sphinganines tested in human prostate adenocarcinoma (prostate cancer) cell line, PC-3. Results show that at least some L-threo-sphinganines increased fenretinide cytotoxicity, additively or synergistically, depending on the drug concentrations tested. Reference is made to the graphs plotting the results shown in FIGS. 43A-43D which results are reported in TABLE 17.1 below.

TABLE 17.1
PC-3

| 4-HPR | L-threo-Sphinganine | C12 | | C14 | | C15 | | C17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.245 | 0.5 | 0.192 | 0.7 | 0.200 | 0.6 | 0.214 | 0.7 | 0.202 | 1.1 | 0.233 | 1.0 | 0.217 | 1.0 | 0.215 | 1.7 |
| 3 | 1 | 0.328 | 0.8 | 0.283 | 1.0 | 0.287 | 0.9 | 0.338 | 0.9 | 0.346 | 1.4 | 0.406 | 1.1 | 0.393 | 1.1 | 0.350 | 1.4 |
| 6 | 2 | 0.526 | 0.9 | 0.426 | 1.3 | 0.417 | 1.2 | 0.623 | 0.8 | 0.719 | 1.1 | 0.635 | 1.1 | 0.620 | 1.2 | 0.479 | 1.6 |
| 9 | 3 | 0.721 | 0.8 | 0.634 | 1.2 | 0.621 | 1.0 | 0.862 | 0.5 | 0.918 | 0.7 | 0.913 | 0.5 | 0.916 | 0.5 | 0.738 | 0.9 |

### Example B18

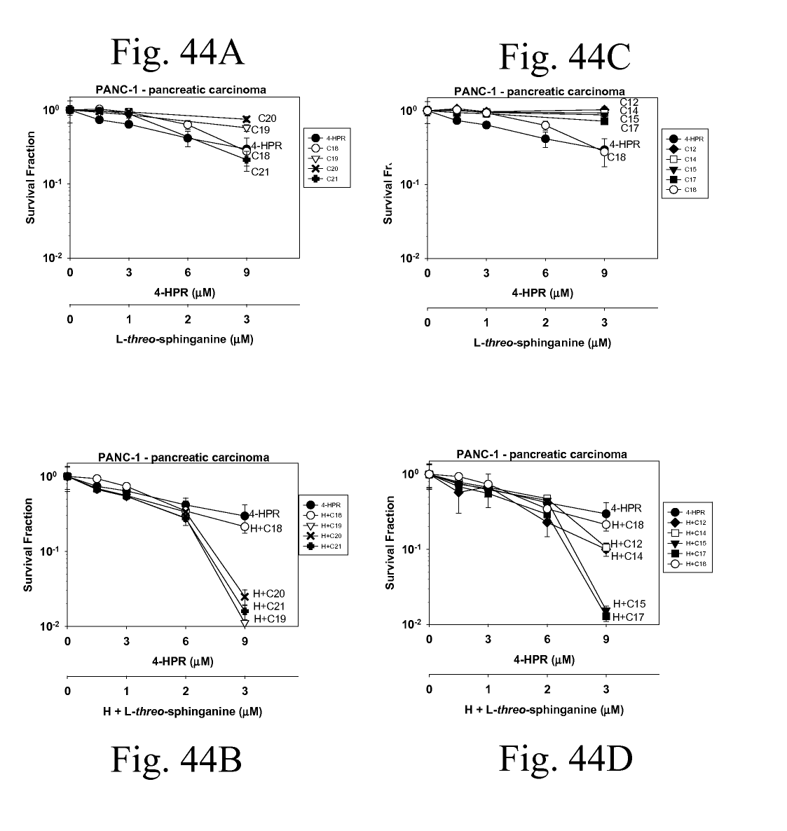

Fenretinide and L-threo-sphinganines tested in human pancreatic adenocarcinoma (pancreas cancer) cell line, PANC-1. Results show that at least some L-threo-sphinganines increased fenretinide cytotoxicity, additively or synergistically, depending on the drug concentrations tested. Reference is made to the graphs plotting the results shown in FIGS. 44A-44D which results are reported in TABLE 18.1 below.

TABLE 18.1
PANC-1

| 4-HPR | L-threo-Sphinganine | C12 | | C14 | | C15 | | C17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |
| 1.5 | 0.5 | 0.232 | 0.9 | 0.054 | 3.2 | 0.078 | 2.9 | 0.171 | 1.5 | 0.136 | 2.5 | 0.182 | 1.5 | 0.136 | 1.9 | 0.111 | 2.5 |
| 3 | 1 | 0.119 | 3.1 | 0.176 | 2.6 | 0.205 | 2.2 | 0.311 | 1.7 | 0.336 | 2.1 | 0.321 | 1.8 | 0.291 | 1.9 | 0.239 | 2.9 |
| 6 | 2 | 0.757 | 0.7 | 0.450 | 2.1 | 0.486 | 1.7 | 0.714 | 0.9 | 0.620 | 1.4 | 0.663 | 1.4 | 0.653 | 1.2 | 0.784 | 1.6 |
| 9 | 3 | 0.977 | 0.2 | 0.883 | 0.7 | 0.978 | 0.2 | 0.990 | 0.1 | 0.700 | 1.5 | 0.997 | 0.1 | 0.990 | 0.1 | 0.994 | 0.4 |

### Example B19

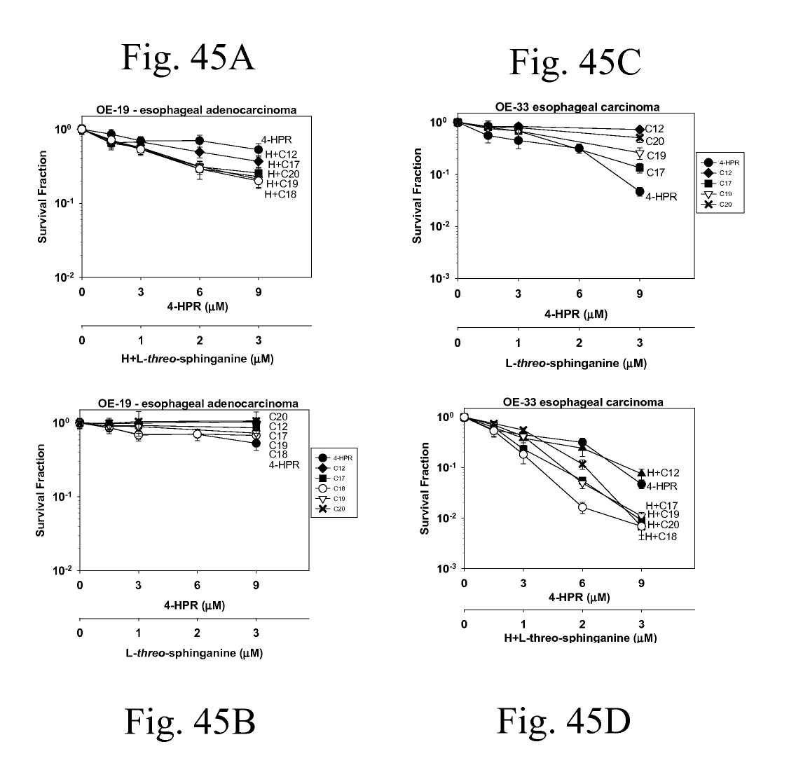

Fenretinide and L-threo-sphinganines tested in human esophageal adenocarcinoma (esophagus cancer) cell lines, OE-19 and OE-33. Results show that at least some L-threo-sphinganines increased fenretinide cytotoxicity, additively or synergistically, depending on the drug concentrations tested. Reference is made to the graphs plotting the results shown in FIGS. 45A-45D which results are reported in TABLES 19.1 and 19.2 below.

TABLE 19.1
OE-19

| 4-HPR | L-threo-Sphinganine | C12 | | C14 | | C15 | | C17 | | C18 | | C19 | | C20 | |
|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl |

| | | | | | | | | | | | |
|---|---|---|---|---|---|---|---|---|---|---|---|
| 1.5 | 0.5 | 0.345 | 1.4 | 0.314 | 0.3 | 0.281 | 0.7 | 0.094 | 1.0 | 0.352 | 3.2 |
| 3 | 1 | 0.325 | 2.9 | 0.462 | 0.3 | 0.464 | 0.4 | 0.109 | 1.6 | 0.438 | 6.6 |
| 6 | 2 | 0.506 | 5.5 | 0.689 | 0.2 | 0.709 | 0.2 | 0.173 | 1.6 | 0.708 | >>1 |
| 9 | 3 | 0.632 | >>1 | 0.743 | 0.2 | 0.799 | 0.2 | 0.272 | 1.1 | 0.771 | >>1 |

TABLE 19.2
OE-33

| 4-HPR | L-threo-Sphinganine | C12 | | C14 | | C15 | | C17 | | C18 | | C19 | | C20 | | C21 | |
|---|---|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl | Fₐ | Cl |

| | | | | | | | | | |
|---|---|---|---|---|---|---|---|---|---|
| 1.5 | 0.5 | 0.376 | 1.0 | 0.376 | 1.5 | 0.299 | 1.9 | 0.255 | 1.9 |
| 3 | 1 | 0.618 | 1.0 | 0.767 | 1.1 | 0.582 | 1.6 | 0.445 | 1.9 |
| 6 | 2 | 0.758 | 1.3 | 0.945 | 0.8 | 0.951 | 0.6 | 0.883 | 0.8 |
| 9 | 3 | 0.925 | 1.1 | 0.991 | 0.4 | 0.989 | 0.3 | 0.993 | 0.2 |

### Example B20

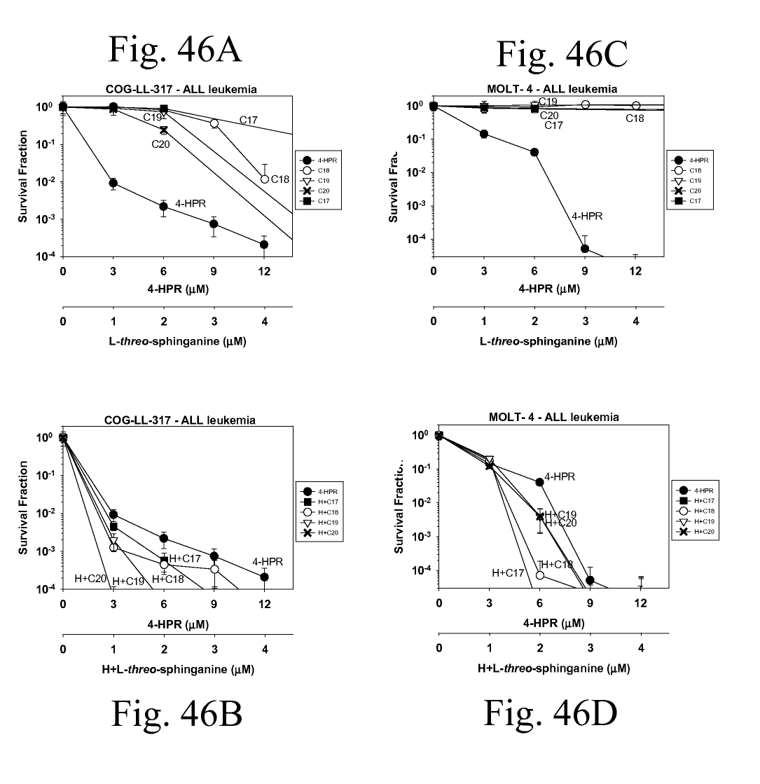

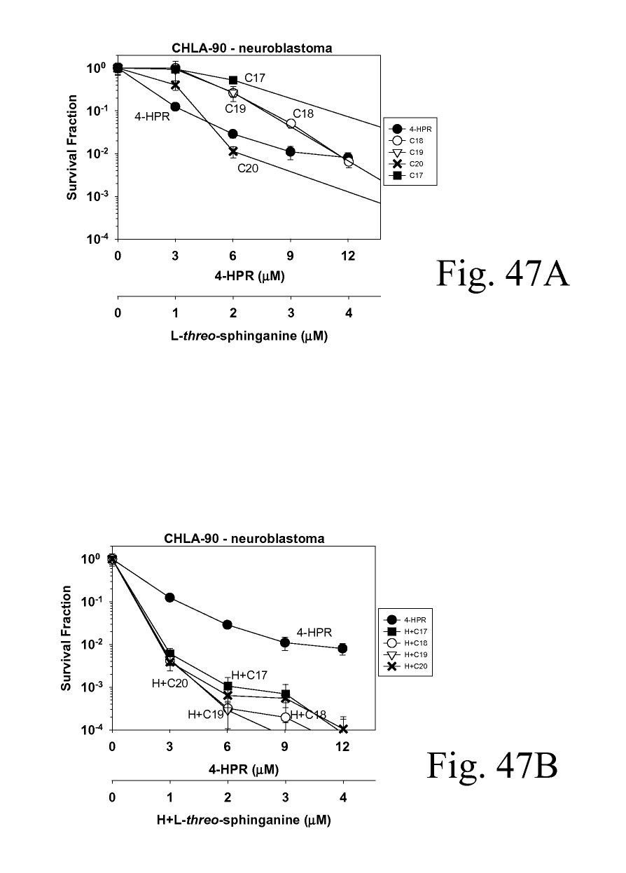

Fenretinide and L-threo-sphinganines tested in human acute lymphoblastic leukemia (pediatric ALL, a blood cancer) cell lines, COG-LL-317 and MOLT-4. Results show that at least some L-threo-sphinganines increased fenretinide cytotoxicity, additively or synergistically, depending on the drug concentrations tested. Reference is made to the graphs plotting the results shown in FIGS. 46A-46D which results are reported in TABLES 20.1 and 20.2 below.

TABLE 20.1
COG-LL-317

| 4-HPR | L-threo-Sphinganine | C12 | | C14 | | C15 | | C17 | | C18 | | C19 | | C20 | |
|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl |

| | | | | | | | | | |
|---|---|---|---|---|---|---|---|---|---|
| 3 | 1 | 0.99300 | 0.8 | 0.99800 | 0.8 | 0.9980 | 0.8 | 0.9990 | 0.2 |
| 6 | 2 | 0.99930 | 1.3 | 0.99940 | 0.6 | 0.9999 | 0.4 | 1.0000 | 0.3 |
| 9 | 3 | 0.99993 | 0.5 | 0.99960 | 0.8 | 1.0000 | na | 1.0000 | na |
| 12 | 4 | 1.00000 | na | 1.00000 | na | 1.0000 | na | 1.0000 | na |

TABLE 20.2
MOLT-4

| 4-HPR | L-threo-Sphinganine | C12 | | C14 | | C15 | | C17 | | C18 | | C19 | | C20 | |
|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl |

| | | | | | | | | | |
|---|---|---|---|---|---|---|---|---|---|
| 3 | 1 | 0.807 | 1.0 | 0.829 | 0.9 | 0.797 | 1.0 | 0.878 | 0.9 |
| 6 | 2 | 0.998 | 0.7 | 0.998 | 0.7 | 0.996 | 0.8 | 0.996 | 0.8 |
| 9 | 3 | 0.999 | 0.8 | 0.999 | 0.8 | 0.999 | 0.8 | 0.999 | 0.8 |
| 12 | 4 | 1 | na | 1 | na | 1 | na | 1 | na |

### Example B21

Fenretinide and L-threo-sphinganines tested in human pediatric neuroblastoma (a nerve-related, solid tumor cancer) cell line, CHLA-90. Results show that at least some L-threo-sphinganines increased fenretinide cytotoxicity, additively or synergistically, depending on the drug concentrations tested. Reference is made to the graphs plotting the results shown in FIGS.

47A-47B which results are reported in TABLE 21.1 below.

TABLE 21.1
CHLA-90

| 4-HPR | L-threo-Sphinganine | C12 | | C14 | | C15 | | C17 | | C18 | | C19 | | C20 | |
|---|---|---|---|---|---|---|---|---|---|---|---|---|---|---|---|
| (μM) | (μM) | Fₐ | Cl | Fₐ | Cl | Fₐ | Cl | Fₐ | Cl | Fₐ | Cl | Fₐ | Cl | Fₐ | Cl |

| | | | | | | | | | |
|---|---|---|---|---|---|---|---|---|---|
| 3 | 1 | 0.99400 | 0.4 | 0.99600 | 0.5 | 0.9960 | 0.4 | 0.996 | 0.5 |
| 6 | 2 | 0.99900 | 0.4 | 0.99930 | 0.3 | 0.9993 | 0.1 | 0.999 | 0.7 |
| 9 | 3 | 0.99970 | 0.6 | 0.99992 | 0.4 | 0.9995 | 0.1 | 0.999 | 1.1 |
| 12 | 4 | 0.99996 | 0.3 | 1.00000 | na | 1.0000 | na | 0.999 | 1.5 |

The foregoing is illustrative of the present invention, and is not to be construed as limiting thereof. The invention is defined by the following claims, with equivalents of the claims to be included therein.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed method, except as may be outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be openended, i.e., to mean including but not limited to. Only the transitional phrases “consisting of” and “consisting essentially of,” respectively, shall be closed or semi-closed transitional phrases.

All of the compositions and methods disclosed and claimed herein can be made and executed without undue experimentation in light of the present disclosure. While the compositions and methods of this invention have been described in terms of preferred embodiments, it will be apparent to those skilled in the art that variations may be applied to the apparatus and/or methods and in the steps or in the sequence of steps of the method described herein without departing from the concept, spirit, and scope of the invention.

More specifically, it will be apparent that certain components which are both shape and material related may be substituted for the components described herein while the same or similar results would be achieved. All such similar substitutes and modifications apparent to those skilled in the art are deemed to be within the spirit, scope, and concept of the invention as defined by the appended claims.

1.-17. (canceled)

18. A method of treating a hyperproliferative disorder in a patient in need of such treatment, the method comprising: intravenously administering a dosage unit of a synergistic amount of a 17- or a 19-carbon L-threo-sphinganine prior to, concurrent with, or subsequent to a ceramide-increasing retinoid or a pharmaceutically acceptable salt thereof, to the patient in need thereof, such that the blood level of L-threo-sphinganines in the patient is raised to about 1 to 4 micromolar plasma levels in the patient, wherein an anti-hyperproliferative activity of the 17- or a 19-carbon L-threo-sphinganine and the ceramide-increasing retinoid is greater than the anti-hyperproliferative activity of either single agent at similar plasma concentrations.

19. The method of claim 18, wherein the blood level of L-threo-sphinganines is raised to about 2 micromolar.

20. The method of claim 18, wherein the blood level of L-threo-sphinganines is raised to about 3 micromolar.

21. The method of claim 18, wherein the blood level of L-threo-sphinganines is raised to about 1.5 to 3.0 micromolar.

22. The method of claim 18, wherein dosage unit is 200-840 milligram/meter-squared per dose.

23. The method of claim 18, wherein the hyperproliferative disorder is a cancer selected from breast, osteosarcoma, angiosarcoma, fibrosarcoma, leukemia, lymphoma, sinus tumor, ovarian, cervical, ureteral, bladder, prostate, genitourinary, colon, esophageal, stomach, gastrointestinal, lung, myeloma, pancreatic, liver, kidney, endocrine, skin, brain, central nervous system, peripheral nervous system, malignant, benign, glioma, or neuroblastoma.

24. The method of claim 18, wherein the ceramide-increasing retinoid is fenretinide.

25. The method of claim 24, wherein the fenretinide is provided orally or intravenously in a dosing sufficient to obtain a plasma level of 8 to 100 micromolar.

26. A method of treating a hyperproliferative disorder in a patient in need of such treatment, the method comprising: orally administering a synergistic dosage unit of a 17 or 19 carbon L-threo-sphinganine and fenretinide to the patient in need thereof such that the blood level of L-threo-sphinganines in the patient is raised to about 1 to 4 micromolar plasma levels in the patient, and wherein a dose is 200-840 milligram/meter-squared, wherein an anti-hyperproliferative activity of the 17- or a 19-carbon L-threo-sphinganine and the ceramide-increasing retinoid is greater than the anti-hyperproliferative activity of either single agent at similar plasma concentrations.

27. The method of claim 26, wherein the blood level of L-threo-sphinganines is raised to about 2 micromolar.

28. The method of claim 26, wherein the blood level of L-threo-sphinganines is raised to about 3 micromolar.

29. The method of claim 26, wherein the blood level of L-threo-sphinganines is raised to about 1.5 to 3.0 micromolar.

30. The method of claim 25, wherein the hyperproliferative disorder is a cancer selected from breast, osteosarcoma, angiosarcoma, fibrosarcoma, leukemia, lymphoma, sinus tumor, ovarian, cervical, ureteral, bladder, prostate, genitourinary, colon, esophageal, stomach, gastrointestinal, lung, myeloma, pancreatic, liver, kidney, endocrine, skin, brain, central nervous system, peripheral nervous system, malignant, benign, glioma, or neuroblastoma.

31. The method of claim 26, wherein the 17 or 19 carbon L-threo-sphinganine is given in a single dose or as a continuous dose by intravenous infusion over 6 to 72 hrs.

32. The method of claim 26, wherein the ceramide-increasing retinoid is fenretinide.

33. The method of claim 32, wherein the fenretinide is provided orally or intravenously in a dosing sufficient to obtain a plasma level of 8 to 100 micromolar.

34. A method of treating a hyperproliferative disorder in a patient in need of such treatment, the method comprising: orally administering a dosage unit of a synergistic amount of:
(i) a 17- or a 19-carbon L-threo-sphinganine, (ii) a glucosylceramide or glucosyl(dihydro)ceramide synthesis inhibitor, and (iii) fenretinide to the patient in need thereof, such that the blood level of L-threo-sphinganines in the patient is raised to about 1 to 4 micromolar plasma levels in the patient and a cytotoxicity of fenretinide is greater than the fenretinide alone, wherein an anti- hyperproliferative activity of the 17- or a 19-carbon L-threo-sphinganine and the fenretinide is greater than the anti-hyperproliferative activity of either single agent at similar plasma concentrations

35. The method of claim 34, wherein the blood level of L-threo-sphinganines is raised to about 2 micromolar.

36. The method of claim 34, wherein the blood level of L-threo-sphinganines is raised to about 3 micromolar.

37. The method of claim 34, wherein the blood level of L-threo-sphinganines is raised to about 1.5 to 3.0 micromolar.

38. The method of claim 34, wherein the hyperproliferative disorder is a cancer selected from breast, osteosarcoma, angiosarcoma, fibrosarcoma, leukemia, lymphoma, sinus tumor, ovarian, cervical, ureteral, bladder, prostate, genitourinary, colon, esophageal, stomach, gastrointestinal, lung, myeloma, pancreatic, liver, kidney, endocrine, skin, brain, central nervous system, peripheral nervous system, malignant, benign, glioma, or neuroblastoma.

39. The method of claim 34, wherein the 17 or 19 carbon L-threo-sphinganine is given in a single dose or as a continuous dose by intravenous infusion over 6 to 72 hrs.

40. The method of claim 34, wherein the glucosylceramide or glucosyl(dihydro)ceramide synthase inhibitor is D-threo-1-phenyl-2-palmitoylamino-3-morpholino-1-propanol.
